(19) United States
(12) Patent Application Publication
(10) Pub. No.: US 2012/0220729 A1
(43) Pub. Date: Aug. 30, 2012

(54) LIQUID PHASE SEPARATION OF PLASMID DNA ISOFORMS AND TOPOISOMERS

(75) Inventors: Michael LAEMMERHOFER, Tuebingen (DE); Wolfgang LINDNER, Klosterneuburg (AT); Marek MAHUT, Laxenburg (AT)

(73) Assignee: BOEHRINGER INGELHEIM INTERNATIONAL GMBH, Ingelheim am Rhein (DE)

(21) Appl. No.: 13/402,166

(22) Filed: Feb. 22, 2012

(30) Foreign Application Priority Data
Feb. 28, 2011 (EP) 11 156 235.1

(51) Int. Cl. C07H 1/06; C08F 265/04; C07F 7/02

(52) U.S. Cl. 525/279; 546/14; 536/25.4

(57) ABSTRACT

The invention relates to the use of material comprising a carbamoyl-decorated anion-exchanger ligand wherein a cationic group (C), and a hydrogen donor group (N—H), are connected by a spacer with a length between 3 to 5 atoms, immobilized onto or embedded into a heterogeneous matrix, for the separation of isoforms, topoisomers, oligomeric and multimeric forms of plasmid DNA (pDNA).

### TECHNICAL FIELD

The present invention relates to the chromatographic materials and methods for separation of isoforms, topoisomers, oligomeric and multimeric forms of plasmid DNA (pDNA) governed by use of carbamoyl-decorated anion-exchange ligands immobilized or embedded into a heterogeneous matrix.

### BACKGROUND ART

Plasmid DNA (pDNA) is an extrachromosomal genetic unit providing its host cell with additional functionalities. Since the discovery of its great potential for use in gene therapy or genetic vaccination, much attention is paid to biotechnological production of these novel type of drugs (Schleef, M., Plasmids for Therapy and Vaccination, Wiley-VCH, Weinheim 2001). From more possible isoforms, the so-called covalently closed circular (ccc) form is considered to be most active for therapeutics. Pharmaceutical grade ccc plasmid DNA is often produced by fermentation in E. coli followed by harvesting, alkaline lysis and multi-step purification of pDNA (Urthaler, J., et al., Journal of Biotechnology, 2007, 128, pgs: 132-149). For human gene therapy, all these steps must meet regulatory guidelines. Therefore the use of animal derived compounds (e.g. enzymes like hen egg white lysozyme) and/or toxic reagents shall be avoided. Furthermore, production processes have to guarantee a low level of host-related impurities (such as endotoxins in the case of bacterial host), see in Urthaler, J., Chemical Engineering & Technology, 2005, 28, pgs: 1408-1420. Additionally, a high content of the ccc form relative to the other pDNA isoforms is recommended in the final formulation of the bioactive drug (FDA, U., in: Administration, F. a. D. (Ed.), Considerations for Plasmid DNA Vaccines for Infectious Disease Indications, Rockville, Md. 2007).

There is a need for a reliable analysis of the plasmid isoform distribution during upstream and downstream processing as well as in the quality control. Apart from the ccc form, other forms need to be monitored which are considered less effective for therapy and vaccination: (a) the open circular form (oc) generated from the ccc form by a single nick in the DNA backbone, and (b) the linear form (lin) created in case when two nicks are in close vicinity. Besides these monomeric forms, also di- and oligomeric species may be generated during fermentation (Lahijani, R. et al., Human Gene Therapy, 1996, 7, pgs: 1971-1980) such as concatemers (i.e. a long continuous DNA molecule that contains multiple copies of the same DNA sequences linked in series) and catenanes (i.e. circular DNA that is interlaced together as links in a chain). The catenated DNA is attached loop to loop in contrast to the concatenated DNA which is attached end to end.

There are various methods available for the effective separation of total plasmid DNA (Stadler J., et al., J Gene Med, 2004; 6, pgs: S54-S66), e.g. thiophilic aromatic chromatography (Sandberg, L. M., et al., Journal of Biotechnology, 2004, 109, pgs: 193-199), hydrophobic interaction chromatography (Ferreira, G. N. M., et al., Pharm. Pharmacol. Commun., 1999, 5, pgs: 57-59; Diogo, M. M., et al., Journal of Chromatography A, 2005, 1069, pgs: 3-22; Tiainen P. et al., Journal of Chromatography A, 2007, 1138, pgs: 84-94; Urthaler J. et al., Chemical Engineering and Technology, 2005, 28, pgs: 1408-1420), or peptide affinity chromatography (Han Y. and Gareth M. F., J. Chromatogr. B, 2008, 874, pgs: 21-26). In EP 1 187 840 B1 by using hydrophobic interaction chromatography (HIC) a preparative separation of two pDNA isoforms, namely ccc and oc was disclosed. However, in this case the separation is based on the non-specific (i.e. hydrophobic) interaction while suffering from the relatively low separation efficiency (e.g. on pg. 33 in FIG. 7) when compared to the process according to the present invention. Furthermore, due to extremely high loads of cosmotropic salts necessary for HIC separation (e.g. on pg. 28 in FIG. 1), the product solutions obtained after such loads need to be desalted accordingly, i.e. abundant salts need to be removed by use of e.g. size exclusion chromatography, dialysis or membrane separation. In addition the cumulated salts' disposal is financially very demanding. In addition, isolation of various types of nucleic acids by use of poly(L-lysine) kieselguhr chromatography was described (e.g. in Finkelstein D. B., et al., J. Biochemistry, 1972, 11, 25, pgs: 4853-8; or in Monaghan-Cannon M. and Dunican L. K., The Biochemical Journal, 1969, 115, 3, 7P). Furthermore, amino acid (Arg, His, Lys) affinity chromatographic separations were disclosed (a) for analytical use in Sousa A., et al., J. Sep. Sci. 2010, 33, pgs: 2610-2618, and (b) for preparative purposes in Sousa F., et al., Trends in Biotechnology, Vol. 26 No. 9, 2008, pgs: 518-525. Recently, a 16-mer peptide with the sequence NH₂-Cys-Met-Lys-Tyr-Val-Ser-His-Gly-Thr-Val-Ser-Arg-Val-Val-Asn-Gln-COON bound to polymethacrylate monolith was reported to show binding to pDNA and selectivity for some isoforms in Y. Han, et al., J. Chromatogr. B, 2008, 874, pgs: 21-26.

Native samples of ccc plasmids consist of a series of individual topological isomers (topoisomers), which differ by various degrees of supercoiling. These ccc topoisomers have different linking numbers, thus a different number of superhelical turns (Depew, R. E., Wang, J. C., Proc. Natl. Acad. Sci. U.S.A., 1975, 72, pgs: 4275-4279). Supercoiling consists of two quantities, twist (Tw) and writhe (Wr), which are interconvertible but for a certain topoisomer the sum of both is invariant. Bacterial plasmids, a key element in biotechnology, molecular biology and the development of novel biopharmaceuticals and therapeutics, are negatively supercoiled in vivo. The amount of supercoiling is around 6%, meaning that on average 6 negative supercoils are introduced per 100 helical turns of the most common type of DNA, so called B-DNA, under standard conditions, corresponding to 6 negative supercoils per 1050 base pairs on average (if the standardized value of 10.5 base pairs per turn is used for the calculation). The topoisomer pattern was found to be changed during bacterial cells proliferation, in vivo according to a circadian rhythm (Woelfle, M. A., PNAS, 2007, 104, 47, pgs: 18819-18824).

Concerning the methods of the plasmid topoisomer separation, there are available various gel-based electrophoretic methods including a capillary gel electrophoresis with the highest resolving power (Schmidt, T., et al., Analytical Biochemistry, 1999, 274, pgs: 235-240), however suffering overall from a low robustness, the low sample salt-tolerance and high time-consumption. The prerequisite of DNA intercalating agent might cause unwanted toxicity within this procedure. Therefore, liquid chromatography based methods are preferred both for analytical and preparative applications. A large number of different chromatographic applications and different chromatographic supports are available for the preparative plasmid purification of various quality preferences (e.g. Diogo, M. M., et al., Journal of Chromatography, A 2005, 1069, pgs: 3-22; Wu, L., Pang, G.-c., Chromatographia 2007, 66, pgs: 151-157; Tiainen, P. et al., Journal of Chromatography, A 2007, 1149, pgs: 158-168; Sousa, F. et al., Analytical Biochemistry 2005, 343, pgs: 183-185; or Urthaler, J., et al., Journal of Chromatography, A 2005, 1065, pgs: 93-106). However, in the analytical scale the choice is limited to anion exchange columns, such as the DNA-NPR from Tosoh Bioscience (Tosoh Corporation, Tokyo, Japan) or GenPak FAX from Waters (Waters Corporation, Milford, Mass., USA), employing the same basis material, i.e. non-porous 2.5 μm particles composed of a hydrophilic organic polymer with diethylaminoethyl (DEAE)-based ligands. Topoisomer distribution can be analyzed so far by electrophoretic techniques only. Apart from that, there are neither materials nor methods (including chromatography) available in the state of the art for the separation of this type of ccc plasmid DNA.

Generally, the effectiveness of any chromatographic method mainly depends on three essentials: (1) a length of separation compartment, e.g. column filled with chromatographic material, (2) retention factor (capacity factor) describing the migration rate of an analyte on a column, and (3) selectivity representing ratio between the retention factors of two species which are to be separated. The most impact of those has the latter one, wherein the selectivity is influenced by characteristics of supportive material (also known as solid phase/matrix/support) and of the nature of the ligand attached to or embedded into the support.

Porous supports with mid-sized pores (mesopores) used for small molecules do not allow the biomolecules to enter inside (Rozing, G. P., Journal of Chromatography A, 1989, 476, pgs: 3-19). Large and gigaporous materials suffer from slow mass transport and low sample recovery. The most promising option in the art for use in analytics are considered to be micropellicular stationary phases characterized by a thin retentive layer at the surface of a non-porous particle (Huber, C. G., Journal of Chromatography, A, 1998, 806, pgs: 3-30). Another alternative (particular suitable for preparative uses) represent monoliths due to their excellent binding capacities and ultra-short analysis with benefits of high resolution at fast column flow and no mass transfer limitation (Hahn, R., et. al., Separation Science and Technology, 2002, 37, pgs: 1545-1565).

In the state of the art less attention is given to the ligands employed in ion exchange chromatography for plasmid separation, wherein simple tertiary (such as DEAE, diethylaminoethyl) or quaternary (often abbreviated with Q) amines are used as a standard. These rather unspecific groups separate nucleic acids according to the number of negative charges of the analyte (Wieder, W., et al., Journal of Separation Science, 2006, 29, pgs: 2478-2484) resulting in relatively insufficient separation. Since the separation, which is highly relevant both for analytical and preparative approach, depends on charge density and hydrodynamic factors (Onishi, Y., et al, Analytical Biochemistry, 1993, 210, pgs: 63-68) effects similar to slalom chromatography (i.e. wherein the progression of the DNA molecules through the closed column follows the flow direction and is like a snake edging, so called “reptation”) can be observed. In this approach the pDNA isomer separation is dependent on the flow-rate and gradient slope (Smith, C. R., et al., Journal of Chromatography, B: Analytical Technologies in the Biomedical and Life Sciences, 2007, 854, pgs: 121-127) causing this system to be less robust and more unpredictable. Furthermore, Molloy et al., Nucleic Acid Research, 2004, 32, e129/121-e129/110 point out certain drawbacks of plasmid separation by using ion exchange chromatography based on DEAE ligands, wherein the calculated content of the oc isoform (impurity) in the ccc pDNA sample was significantly lower compared to the application of agarose gel electrophoresis.

Various cinchonan alkaloid ligands including cinchonan carbamates and cinchonan ureas were disclosed as chiral catalysts in asymmetric organic synthesis (WO92/20677 and U.S. Pat. No. 6,197,994B1), and as chiral discriminants (i.e. chiral selectors) in analytical chemistry (Laemmerhofer M., et al., Journal of Chromatography, A 1996, 741, pgs: 33-48; WO97/46557; U.S. Pat. No. 6,616,825 B1; Lee Y.-K. et al., Polymer 43, 2002, pgs: 7539-7547). In addition, amino acid (arginine, lysine and histidine) ligand structures have been currently described in Sousa F., Queiroz J. A., J of Chromatography A, doi:10.1016/j.chroma.2010.11.002 (in press) and Sousa A, et al., J. Sep. Sci. 2010, 33, pgs: 2610-2618, for the separation of oc and ccc pDNA isoforms in the analytical scale.

Overall, sufficiently robust, predictable, reliable and accurate methods and/or materials for the effective separation of all three pDNA isoforms (ccc, oc, and lin) by use of chromatography (including affinity chromatography) both in preparative and analytical scale are not available in the state of the art.

### SUMMARY OF THE INVENTION

The invention relates to use of a material comprising a ligand according to formula 1

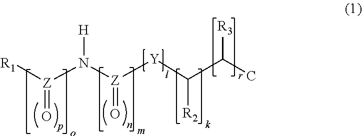

wherein the ligand contains a cationic group (C) and a hydrogen donor group

(N—H) connected by a spacer with a length between 3 to 5 atoms,

m, o and r are independently from one another either 0 or 1, and

k, l, n and p are independently from one another either 0, 1 or 2, and

Y is any moiety selected from the group —CH₂—, —O—, —NH— or —S—,

Z is any moiety selected from the group —C—, —S—, or —P—, and

R₁, R₂ and R₃ are anyone of the substituents from the group consisting of hydrogen, C₁₋₁₈ branched or unbranched alkyl, C₂₋₁₈ branched or unbranched alkenyl, C₂₋₁₈ branched or unbranched alkynyl, C₃₋₁₁-carbocycle, C₃₋₈-heterocycle, C₅₋₁₈-aryl and C₅₋₁₃-heteroaryl,

wherein R₁, R₂ and R₃ optionally comprise independently from one another one or more moieties selected from the group —S—, —O—, —NH—, and

each of R₁, R₂, R₃ can be optionally and independently substituted with one or more substituents selected from the group consisting of hydrogen, C₁₋₆ branched or unbranched alkyl, C₂₋₆ branched or unbranched alkenyl, C₂₋₆ branched or unbranched alkynyl, C₃₋₈-carbocycle, C₃₋₈-heterocycle and C₅₋₁₀-aryl, C₅₋₁₀-heteroaryl, and

whereby the cationic group C is a C₃₋₁₁ heterocycle comprising at least one nitrogen atom and optionally another heteroatom selected from the group consisting of sulphur, oxygen, nitrogen and phosphorus, or

C₅₋₁₈ heteroaryl comprising at least one nitrogen atom and optionally another heteroatom selected from the group consisting of sulphur, oxygen, nitrogen and phosphorus, or

branched or unbranched C₁₋₁₀ alkyl, branched or unbranched C₂₋₁₀ alkenyl, branched or unbranched C₂₋₁₀ alkynyl comprising at least one nitrogen atom and optionally another heteroatom selected from the group consisting of sulphur, oxygen, nitrogen and phosphorus, wherein C and R₃ or C and R₂ are optionally linked to each other by forming a ring, and

wherein the ligand is optionally immobilized onto or embedded into a matrix via R₁ or R₃ group,

for the separation of plasmid DNA isoforms or topoisomers.

Still further, the invention relates to the methods for separation of plasmid DNA isoforms including covalently closed circular (ccc), open circular (oc) and linear (lin) forms, oligomeric and multimeric forms such as monomers, dimers, trimers, tetramers, oligomers, multimers, and/or ccc topoisomers by using of the materials according to the invention.

### BRIEF DESCRIPTION OF FIGURES

FIGS. 1a to 1c: Chromatograms recorded by UV absorption at 258 nm analyzing pDNA samples containing a mixture of ccc with oc and lin isoforms of the pMCP1 plasmid (4.9 kbp) during identical elution conditions.

Three columns are employed, each containing a tert-butylcarbamoyl-quinine ligand attached to different silica matrixes (see structure f), namely 1.5 μm non-porous particles (FIG. 1a), 10 μm porous particles (FIG. 1b) and a monolith (Chromolith Si Performance, Merck) (FIG. 3c). Elution conditions: Method 1 (see Example 4). “Y” axis reflects concentration of pDNA isoforms detected as UV absorption at 258 nm in milliabsorption units [mAU] in relation to “X” axis corresponding to retention time [min].

FIG. 2: Chromatogram recorded by UV absorption at 258 nm from pDNA samples containing ccc, oc and lin isoforms of the pMCP1 plasmid (4.9 kbp) during pH- and 2-propanol mediated gradient elution conditions.

The three main peaks correspond to the three individual pDNA isoforms oc, lin, ccc (as denoted in the FIG. 2). Elution conditions: Method 2. “Y” axis reflects concentration of pDNA isoforms detected as UV absorption at 258 nm in milliabsorption units [mAU] in relation to “X” axis corresponding to retention time [min].

FIGS. 3a and 3b: Chromatograms recorded by UV absorption at 258 nm analyzing pDNA samples with a high ccc content of the pMCP1 plasmid (4.9 kbp) during NaCl-mediated elution.

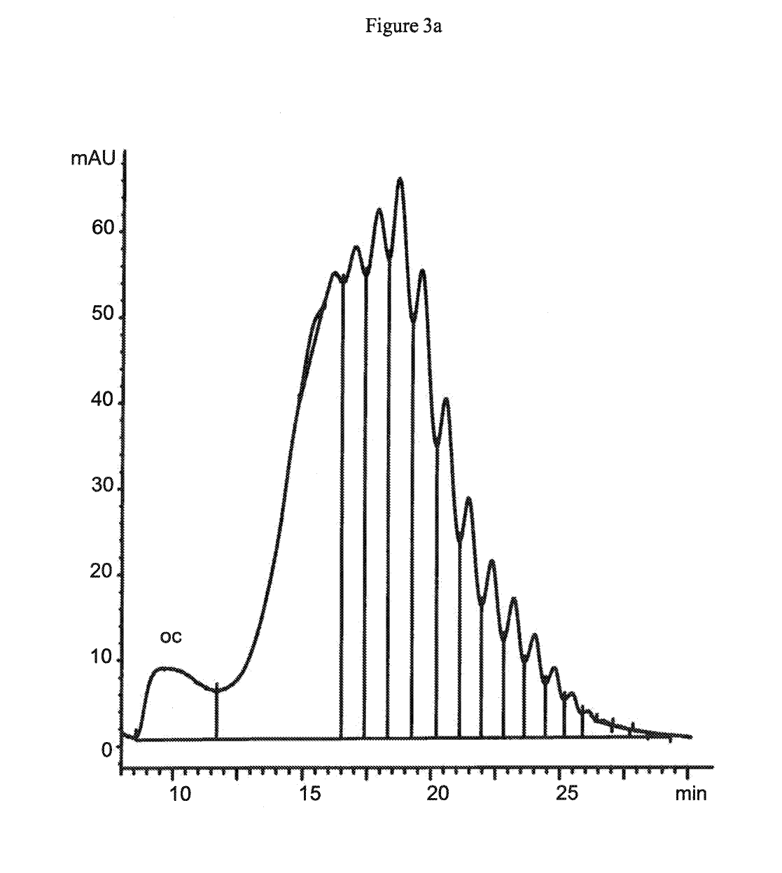

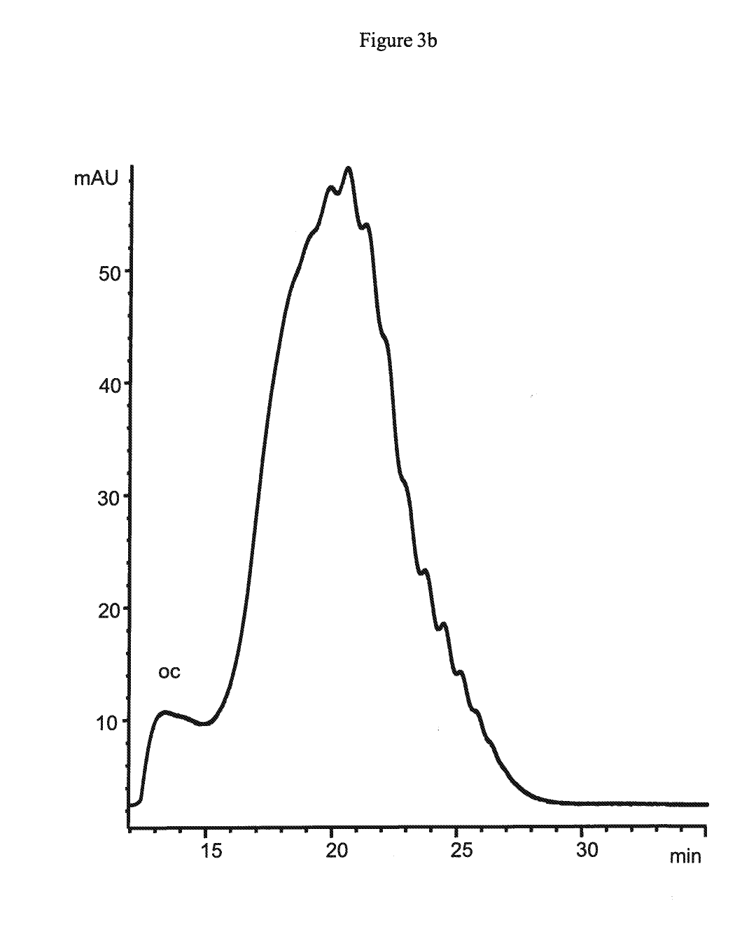

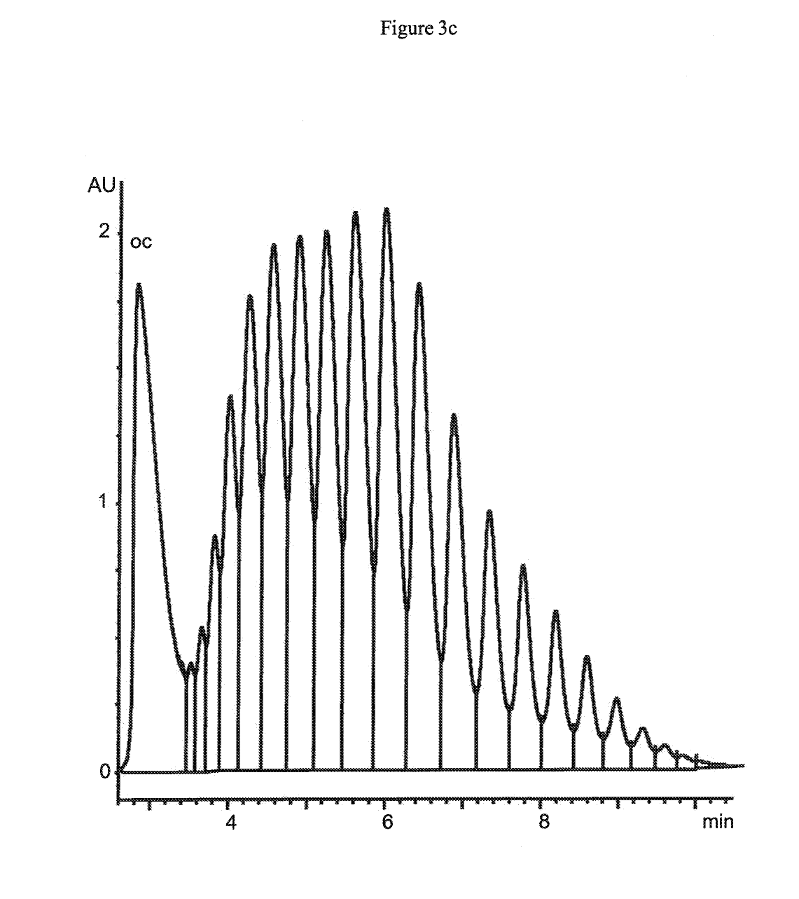

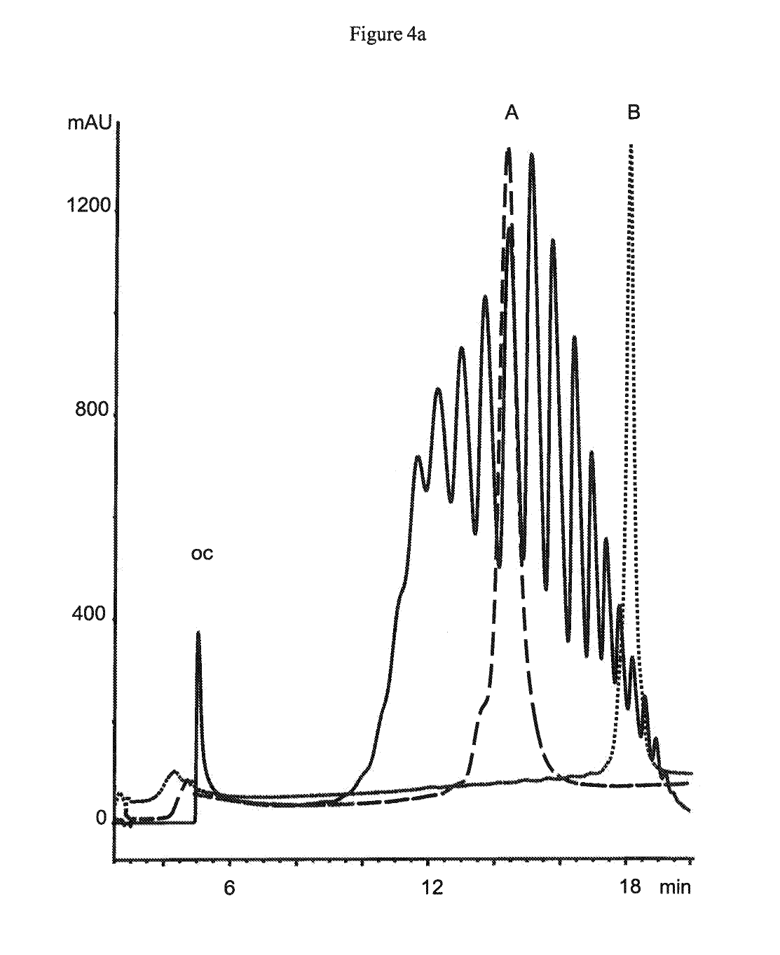

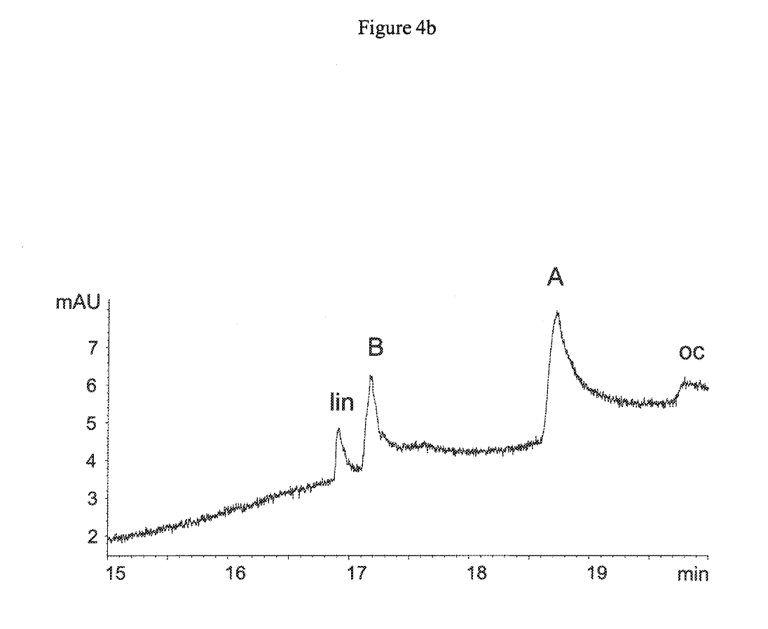

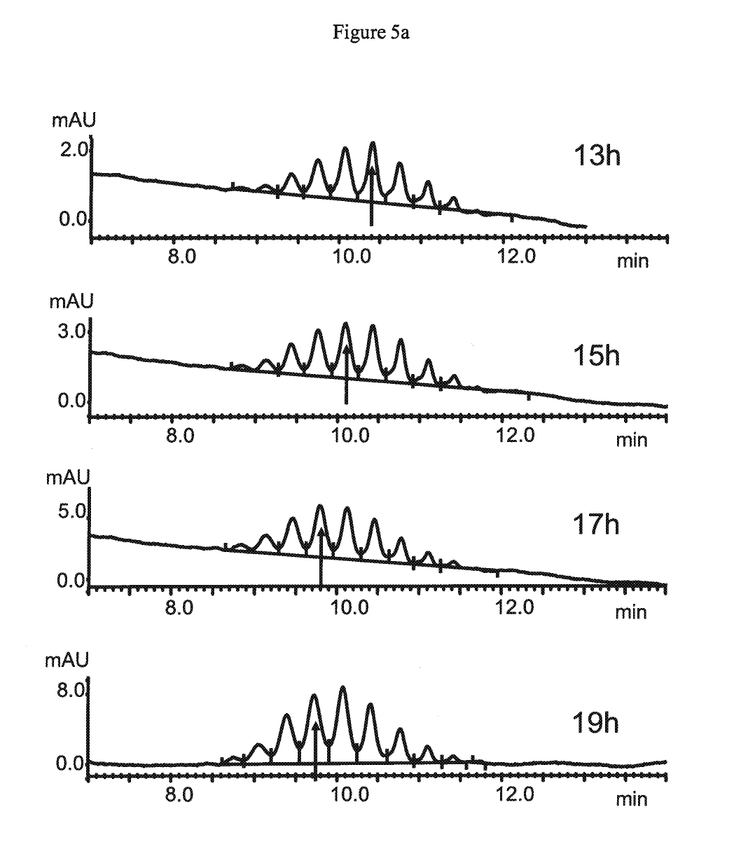

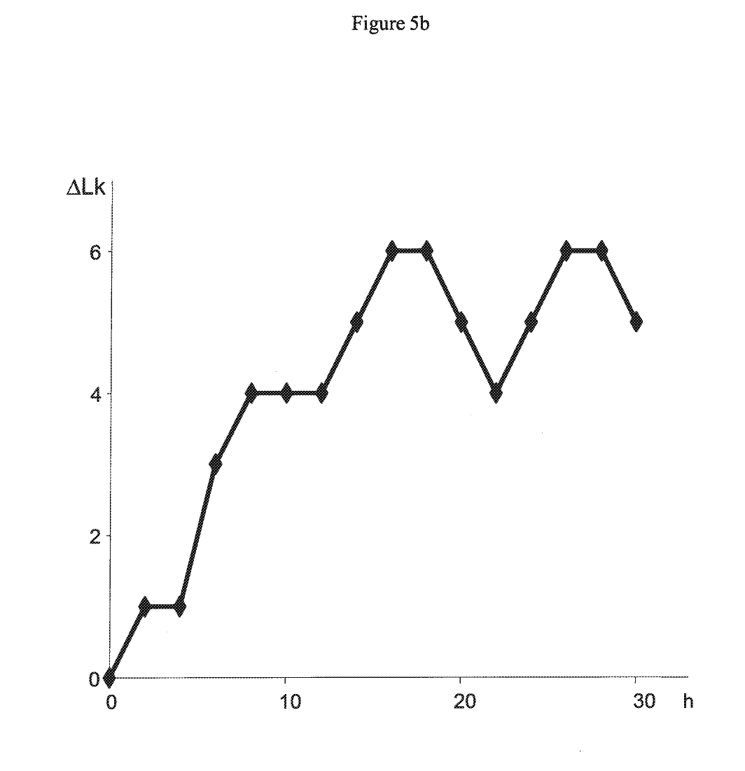

The ccc form in these chromatograms, eluting after the oc isoform, is split into a set of individual topoisomers. The resolution between the topoisomers is higher when using the tert-butylcarbamoyl-quincorine ligand immobilized to 3-mercaptopropyl-modified silica shown in the structure d (FIG. 3a) than when using the tert-butylquincorinyl-urea ligand immobilized to 3-mercaptopropyl-modified silica shown in structure e (FIG. 3b). “Y” axis reflects concentration of pDNA isoforms detected as UV absorption at 258 nm in milliabsorption units [mAU] in relation to “X” axis corresponding to retention time [min].

FIG. 3c: Chromatogram recorded by UV absorption at 258 nm after loading 284 μg of a pDNA samples with a high ccc content of the pMCP1 plasmid (4.9 kbp). Elution is accomplished via NaCl gradient employing a triethoxysilyl-activated propylcarbamoylquinine-ligand bound to 1.5 μm non-porous silica.

The first peak represents the open circular (oc) isoform, while 21 topoisomers of the ccc isoform elute afterwards. “Y” axis reflects concentration of pDNA isoforms detected as UV absorption at 258 nm in absorption units [AU] in relation to “X” axis corresponding to retention time [min].

FIG. 4a: Overlay of chromatograms recorded by UV absorption at 258 nm analyzing pDNA samples with a high ccc content of the pMCP1 plasmid (4.9 kbp) during NaCl-mediated elution employing a triethoxysilyl-activated propylcarbamoylquinine-ligand bound to 1.5 μm non-porous silica.

The ccc form in these chromatograms, eluting after the oc isoform, is split into a set of individual topoisomers. A and B represent isolated topoisomer fractions. The dashed lines stand for fractions A and B reinjected after previous fractionation of the eluted topoisomers on the same column. “Y” axis reflects concentration of pDNA isoforms detected as UV absorption at 258 nm in milliabsorption units [mAU] in relation to “X” axis corresponding to retention time [min].

FIG. 4b: Capillary electrophorectic separation of the mixturecontaining fractions A and B, wherein the fractions are identical to A and B shown in FIG. 4a.

The linear form is the fastest migrating form. Fraction B migrates faster than fraction A, thus indicating its smaller size and higher supercoiling. “Y” axis reflects concentration of pDNA isoforms detected as UV absorption at 258 nm in milliabsorption units [mAU] in relation to “X” axis corresponding to migration time [min].

FIG. 5a: An overlay of chromatograms recorded by UV absorption at 258 nm from pDNA samples drawn during pDNA fermentation is obtained after 11, 13, 15 and 17 hrs by employing the material disclosed as the structure b during NaCl-mediated gradient elution in the second chromatographic dimension, i.e. upon purification step of crude samples.

The arrow marks the highest abundant topoisomer within the topoisomer distribution and thus serves as an indicator of the overall supercoiling, obviously changing during fermentation. In all chromatograms with exception of FIG. 4b, “Y” axis reflects concentration of pDNA isoforms detected as UV absorption at 258 nm in milliabsorption units [mAU] in relation to “X” axis corresponding to retention time [min].

FIG. 5b: The maximum of the topoisomer distribution (calculated as maximum peak area) denoted as a relative linking number of the highest abundant topoisomer which is a measure of the overall supercoling (at the beginning of fermentation this value is zero) (y axis) is plotted against the entire duration of the fermentation (x axis).

Overall the topoisomer pattern changes during fermentation. “Y” axis stands for the relative linking number ΔLk in relation to “X” axis reflecting the duration time [hours].

### DETAILED DESCRIPTION OF THE INVENTION

It has now unexpectedly been found that a material according to the invention comprising a carbamoyl-decorated anion exchange ligand immobilized or embedded into a heterogeneous matrix can be used for the separation of isoforms, topoisomers, oligomeric and multimeric forms of plasmid DNA (pDNA).

In its broadest aspect, the invention provides a material which comprises a ligand according to formula 1

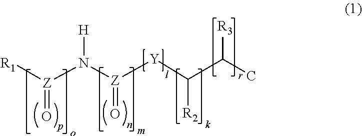

wherein the ligand contains a cationic group (C) and a hydrogen donor group (N—H) connected by a spacer with a length between 3 to 5 atoms,

m, o and r are independently from one another either 0 or 1, and

k, l, n and p are independently from one another either 0, 1 or 2, and

Y is any moiety selected from the group —CH₂—, —O—, —NH— or —S—,

Z is any moiety selected from the group —C—, —S—, or —P—, and

R₁, R₂ and R₃ are anyone of the substituents from the group consisting of hydrogen, C₁₋₁₈ branched or unbranched alkyl, C₂₋₁₈ branched or unbranched alkenyl, C₂₋₁₈ branched or unbranched alkynyl, C₃₋₁₁-carbocycle, C₃₋₈-heterocycle, C₅₋₁₈-aryl and C₅₋₁₃-heteroaryl,

wherein R₁, R₂ and R₃ optionally comprise independently from one another one or more moieties selected from the group —S—, —O—, —NH—, and

each of R₁, R₂, R₃ can be optionally and independently substituted with one or more substituents selected from the group consisting of hydrogen, C₁₋₆ branched or unbranched alkyl, C₂₋₆ branched or unbranched alkenyl, C₂₋₆ branched or unbranched alkynyl, C₃₋₈-carbocycle, C₃₋₈-heterocycle and C₅₋₁₀-aryl, C₅₋₁₀-heteroaryl, and

whereby the cationic group C is a C₃₋₁₁ heterocycle comprising at least one nitrogen atom and optionally another heteroatom selected from the group consisting of sulphur, oxygen, nitrogen and phosphorus, or

C₅₋₁₈ heteroaryl comprising at least one nitrogen atom and optionally another heteroatom selected from the group consisting of sulphur, oxygen, nitrogen and phosphorus, or

branched or unbranched C₁₋₁₀ alkyl, branched or unbranched C₂₋₁₀ alkenyl, branched or unbranched C₂₋₁₀ alkynyl comprising at least one nitrogen atom and optionally another heteroatom selected from the group consisting of sulphur, oxygen, nitrogen and phosphorus, wherein C and R₃ or C and R₂ are optionally linked to each other by forming a ring, and

wherein the ligand is optionally immobilized onto or embedded into a matrix via R₁ or R₃ group,

for the separation of plasmid DNA isoforms or topoisomers.

One of further preferred embodiments represents the material according to formula 2

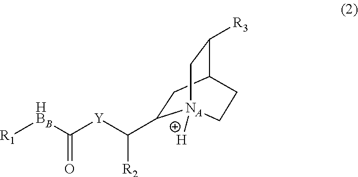

wherein N_{A} represents the nitrogen atom of the cationic group,

N_{B}—H the hydrogen donor (located adjacent to a carbonyl C═O),

Y is an oxygen (—O—) thus yielding a carbamate group or an (—NH—) thus yielding a urea group, and

R₁, R₂, R₃ are anyone of the substituents selected from the group consisting of hydrogen, C₁₋₁₈ branched or unbranched alkyl, C₂₋₁₈ branched or unbranched alkenyl, C₂₋₁₈ branched or unbranched alkynyl, C₃₋₁₁-carbocycle, C₃₋₈-heterocycle, C₅₋₁₈-aryl and C₅₋₁₃-heteroaryl,

wherein R₁, R₂ and R₃ optionally comprise independently from one another one or more moieties selected from the group —S—, —O—, —NH—, and wherein R₁, R₂ and R₃ can be optionally and independently substituted with one or more substituents selected from the group consisting of hydrogen, C₁₋₆ branched or unbranched alkyl, C₂₋₆ branched or unbranched alkenyl, C₂₋₆ branched or unbranched alkynyl, C₃₋₈-carbocycle, C₃₋₈-heterocycle and C₅₋₁₀-aryl, C₅₋₁₀-heteroaryl, and

wherein the substitution optionally comprises the anchoring to a matrix via R₁ or R₃,

for the separation of plasmid DNA or topoisomers.

In one of the most preferred embodiments according to formula 2, Y═—O—, R₁ is alkyl immobilized to silica or thiolpropyl-modified silica, R₂ is methoxyquinoline and R₃ is ethyl or vinyl.

The term “material” according to the invention refers to and comprises a carbamoyl-decorated anion exchange type of ligands optionally immobilized onto or embedded into a heterogeneous (solid or liquid) matrix such as a chromatographic support, substrate, membrane or liquid phase.

The term “carbamoyl” within the meaning of the present invention refers to a moiety selected from a group of —O—C(═O)—NH—, —C(═O)—NH—, —C(═O)—NH₂, or which may also be part of a urea —NH—C(═O)—NH— or carbazide —NH—NH—C(═O)—NH—.

Carbamoyl in the meaning of the present invention does not preclude other isosteric functional groups consisting of a hydrogen-donor acceptor system such as sulphonamide, phosphonamide and similar functionalities, as defined by the formula 2.

The term “ligand” according to the invention refers to and comprises a chemical structure, which is bound via a linker or embedded into a matrix and is able to interact with plasmid DNA. The preferred embodiment of the ligand is quincorine- or quincoridine carbamate, or quincorine- or quincoridine urea. In context of the use for the separation of plasmid DNA isoforms, topoisomers, oligomeric and multimeric forms one of the more preferred embodiments is cinchonan carbamate or cinchonan urea, most preferred is quinine- or quinidine carbamate, in particular propyl carbamoyl quinine or quinidine, tert-butylcarbamoyl-quinine or quinidine, and allylcarbamoyl-10,11-dihydroquinine or quinidine.

The term “optionally substituted” is intended to mean that a hydrogen atom of a methylene group or a hydrogen atom of an aromatic or heteroaromatic ring is substituted with a different organic or inorganic substituent.

The term “C₁₋₁₈-alkyl” herein means a saturated branched or unbranched hydrocarbon chain of 1-18 carbon atoms.

The term “C₁₋₆-alkyl” herein means a saturated branched or unbranched hydrocarbon chain of 1-6 carbon atoms.

The term “C₂₋₁₈-alkenyl” herein means an unsaturated branched or unbranched hydrocarbon chain of 2-18 carbon atoms, which comprises at least one double bond.

The term “C₂₋₆-alkenyl” herein means an unsaturated branched or unbranched hydrocarbon chain of 2-6 carbon atoms, which comprises at least one double bond.

The term “C₂₋₁₈-alkynyl” is intended to mean an unsaturated branched or unbranched hydrocarbon chain of 2-18 carbon atoms, which comprises at least one triple bond.

The term “C₂₋₆-alkynyl” herein is an unsaturated branched or unbranched hydrocarbon chain of 2-6 carbon atoms, which comprises at least one triple bond.

The term “branched” is intended to mean that at least one atom of the hydrocarbon chain, optionally comprising a chain atom is selected from sulphur, oxygen or nitrogen, is covalently bound to another chain atom.

The term “unbranched is intended to mean that any atom of the hydrocarbon chain, optionally comprising a chain atom is selected from sulphur, oxygen or nitrogen, is not covalently bound to any other chain atom.

The term “chain atom” within the meaning of the present invention relates to an atom that exhibits two separate bonds, such as carbon, sulphur, oxygen or nitrogen.

The term “C₃₋₁₁-carbocycle” is intended to mean an aliphatic hydrocarbon 3-11 carbon ring atoms.

The term “C₃₋₈-carbocycle” is intended to mean an aliphatic hydrocarbon 3-8 carbon ring atoms.

The term “C₃₋₈-heterocycle” is a carbocycle comprising 3-8 ring atoms, where at least one of the ring atoms is not carbon but an atom selected from N, O, or S.

The term “C₅₋₁₈-aryl” is an aromatic hydrocarbon ring with 5-18 ring atoms.

The term “C₅₋₁₀-aryl” is an aromatic hydrocarbon ring with 5-10 ring atoms.

The term “C₅₋₁₃-heteroaryl” is an aromatic ring with 5-13 ring atoms, wherein at least one ring atom is not carbon but an atom selected from N, O or S.

The term “C₅₋₁₀-heteroaryl” is an aromatic ring with 5-10 ring atoms, wherein at least one ring atom is not carbon but an atom selected from N, O or S.

In one of the preferred embodiments R₁ is anyone of the substituents from the group consisting of hydrogen, C₁₋₈ branched or unbranched alkyl, C₂₋₈ branched or unbranched alkenyl and C₂₋₈ branched or unbranched alkynyl, optionally comprising one or more sulphur atoms.

In another preferred embodiment R₁ is anyone of the substituents from the group consisting of C₃₋₈-carbocycle, C₅₋₈-heterocycle and C₅₋₁₀-aryl, C₅₋₉-heteroaryl.

In still further preferred embodiment R₃ is anyone of the substituents selected from the group consisting of hydrogen, C₁₋₈ branched or unbranched alkyl, C₂₋₈ branched or unbranched alkenyl and C₂₋₈ branched or unbranched alkynyl, optionally comprising one or more sulphur atoms.

In another preferred embodiment R₂ is anyone of the substituents selected from the group consisting of hydrogen, C₅₋₁₀-aryl and C₅₋₉-heteroaryl.

In one of the most preferred embodiments according to formula 1, o=0 and m=1, or o=1 and m=0.

In still further preferred embodiments according to formula 1, o=0 and m=1, and k=1 and l=1, Z is —C— and n=1. Alternatively, in another most preferred embodiment o=1 and m=0, and k=1 and l=1, Z is —C— and p=1.

In further preferred embodiments according to formula 1, o=0 and m=1, and k=1 and l=1, Z is —S— and n=2.

In still preferred embodiment according to formula 1, o=1 and m=0, k=1 and l=1, Z is —S— and p=2.

The term “anchoring to a matrix” is intended to mean any substituent that forms a stable connection between the ligand and the matrix or between the ligand and residues that are attached to the matrix.

The term “molecular weight” according to the invention refers to the sum of the relative atomic masses of the constituent atoms of the ligand according to the invention. In a preferred embodiment the molecular weight of the ligand is between 200 to 2000 Da, more preferably between 250 to 1200 Da, most preferably between 400 to 700 Da.

The “cationic group C” is a C₃₋₁₁ heterocycle comprising at least one nitrogen atom and optionally another heteroatom selected from the group consisting of sulphur, oxygen, nitrogen and phosphorus, or

C₅₋₁₈ heteroaryl comprising at least one nitrogen atom and optionally another heteroatom selected from the group consisting of sulphur, oxygen, nitrogen and phosphorus, or

branched or unbranched C₁₋₁₀ alkyl, branched or unbranched C₂₋₁₀ alkenyl, branched or unbranched C₂₋₁₀ alkynyl comprising at least one nitrogen atom and optionally another heteroatom selected from the group consisting of sulphur, oxygen, nitrogen and phosphorus,

wherein C and R₃ or C and R₂ are optionally linked to each other by forming a ring.

The term “ring” within the meaning of the invention refers to mono-, bi-, polycyclic or spirocyclic rings.

In a further preferred embodiment the cationic group is incorporated into a cyclic or bicyclic ring system.

In a preferred embodiment the bicyclic ring system is tropane, (hydro)quinolizidine, (hydro)pyrrolizidine, 1-aza-adamantane, azabicyclo[3.2.1]octane, azabicyclo[3.2.1]nonane, azabicyclo[3.3.1]nonane, (hydro)indolizidine, (hydro)benzimidazol, (hydro)quinoline, (hydro)isoquinoline, more preferred azabicyclo[4.4.0]decane, most preferred quinuclidine.

The term “cationic group” within the meaning of the present invention further refers to a moiety containing one nitrogen atom bearing a partial or permanent positive charge selected from a group consisting of tertiary-, quaternary-, secondary-, primary amine or guanidine, amidine and (hetero)aromatic amino group.

A preferred embodiment of the cationic group represents aliphatic amine, more preferred quaternary amine, most preferred tertiary amine. In one of the most preferred embodiments the tertiary amine is a part of quinuclidine.

The term “hydrogen donor moiety” refers to a combination of a hydrogen atom bound to an electronegative atom such as nitrogen, oxygen or fluorine.

The term “hydrogen acceptor group” refers to an electronegative atom such as oxygen, nitrogen, fluorine or sulphur with at least one free electron pair.

The term “spacer” within the meaning of the present invention refers to a group of adjacent atoms of a specified number being bound on either side to functionally important entities, namely a cationic group (C) on one side and a hydrogen donor moiety (N—H) on the other side. The number of adjacent atoms is counted as a shortest distance through an atom chain. The length of the spacer is between 3 and 5 atoms. In the most preferred embodiment the length of the spacer is equal to 4 atoms.

In another more preferred embodiment the ligand according to the invention is located not further than 6 atoms from the matrix.

The term “immobilized” within the meaning of the present invention refers to a covalent bonding onto a solid or liquid matrix (support).

The term “embedded” within the meaning of the present invention refers to a non-covalent inclusion into the solid or liquid matrix (support).

The term “anchoring to a matrix” is intended to mean any substituent that forms a stable connection between the ligand and the matrix or between the ligand and residues that are attached to the matrix.

The term “heterogeneous matrix” within the meaning of the present invention relates to a matrix (support) that cannot be unified with the solvent containing dissolved plasmid DNA being either insoluble or immiscible.

The term “plasmid DNA” refers to an extrachromosomal genetic unit consisting of double stranded deoxyribonucleic acid.

The term “isoforms” within the meaning of the present invention refers to the different forms of plasmid DNA of equal molecular weight selected from the group of covalently closed circular (ccc), open circular (oc) and linear (lin) plasmid. The associated analysis of such isoforms should be denoted as isoform analysis.

The terms “oligomeric and multimeric” within the meaning of the present invention relate to different forms of plasmid DNA of different molecular weight originating from the combination of monomeric isoforms into a larger molecule, the molecular weight of which is an integral multiple of that of the monomer. Subsequently, the term oligomeric marks combinations of two, three or four monomers, while the term multimeric marks combinations of five and more monomers. The oligomeric and multimeric combinations may be either in form of catenane or concatemer.

The term “catenane” according to the invention refers to circular DNA that is interlaced together as links in a chain.

The term “concatemer” according to the invention refers to long continuous DNA molecule that contains multiple copies of the same DNA sequences linked in series.

The terms “catenane” and “concatemer” are characterized in that catenated DNA is attached loop to loop in contrast to concatenated DNA which is attached end to end.

The term “topoisomers” within the meaning of the present invention refers to a covalently closed circular (ccc) plasmid DNA molecules with a different degree of supercoiling, described by the topological linking number or linking number difference. Topoisomers of one individual plasmid have equal molecular weight and connectivity. The associated analysis of such topoisomers should be denoted as topology analysis or topoisomer analysis.

The term “separation” within the meaning of the present invention relates to a segregation of different forms of plasmid DNA such as isoforms, topoisomers, oligomeric and multimeric forms based on different affinities of these forms to the ligand (referred to as chemoaffinity principle).

The term “chromatography” refers to a set of separation techniques employing two heterogeneous phases, while one phase, the so called mobile phase, is moving along the other phase, the so called stationary phase.

Preferred embodiment is the material according to the invention wherein the hydrogen donor N—H is located adjacent to a hydrogen acceptor group.

The term “adjacent” in the meaning of the present invention denotes a direct connection between two atoms or atom groups by a single covalent bond.

In the preferred embodiment the hydrogen acceptor is selected from carbonyl (C═O), sulfonyl (SO₂) and phosphonyl group (P═O).

In still further preferred embodiment the hydrogen acceptor and hydrogen donor group form part of amide, carbamate, urea, phosphonamide or sulphonamide group.

In further preferred embodiment the plasmid DNA comprises mixtures of any components selected from the group consisting of ccc and its topoisomers, oc, lin, dimer, trimer, tetramer, oligomer, multimer (in form of catenane or concatemer) independent from the size of the plasmid and its nucleotide sequence.

Within the meaning of the present invention the term “matrix” encompasses a solid support capable of attaching a ligand, or a liquid capable of dissolving the ligand.

The term “solid matrix” within the meaning of the present invention refers to a solid support suitable for the desired chromatographic separation or liquid-solid extraction, selected from a group of inorganic polymers or organic polymers, preferably micropelicullar silica-based particles.

The term “micropelicullar” refers to a non-porous matrix particle which is characterized by a core of fluid-impervious support material (fused-silica particles or organic polymer microspheres) covered by a thin, retentive layer of stationary phase. The thin retentive layer of stationary phase can be obtained by roughening the surface and attaching the respective chromatographic ligand to obtain an adsorption surface that can retain the target solutes.

The term “liquid matrix” refers to a liquid selected from a group of apolar solvents such as alkanes, cycloalkanes, or polymeric liquids such as polysiloxanes, polyethyleneoxides, or a polymeric liquid such as polysiloxane or polyethylenoxide diluted in another solvent.

In a preferred embodiment the matrix according to the invention is selected from organic or inorganic polymers.

The term “organic polymers” refers to a solid organic polymer such as poly(meth)acrylate and its copolymers, polystyrene and its copolymers, poly(meth)acrylamide and its copolymers, ring-opening methathesis polymers, polysaccharides and its copolymers such as agarose, and bears chemical groups at the polymer surface capable of a covalent attachment of a spacer and/or a ligand.

The term “inorganic polymers” refers to a solid inorganic polymer such as silicon oxide (silica), titanium oxide, germanium oxide, zirconium oxide, aluminium oxide and glass. The polymer surface is capable of a covalent attachment of a spacer and/or a ligand.

In still further preferred embodiment the solid matrix is a particle, a monolith resin, a membrane, or a magnetic bead.

The term “particle” refers to a solid matrix structure of various sizes and morphological features including but not limited to irregular, regular, spherical, porous, non-porous, micropellicular. Usually there is a large number (>1000) of individual particles within one separation compartment.

The term “monolith resin” refers to a solid matrix structure containing flow-through channels allowing for convective flow, also called macropores, optionally containing smaller-sized pores additionally such as mesopores and micropores. Usually there is a single piece or only a small number (<10) of individual monolith resins in one separation compartment.

The term “membrane” refers to a solid matrix in the shape of a thin layer being permeable for the solvent and semi-permeable for the solutes include the sample components. The membrane is selected from the group of polysaccharides such as cellulose, cellulose ester, poly(meth)acrylates, poly(styrenes), polytetrafluoroethylene, polysulfone, polyacrylonitril, polyphenylenoxide, polyethylene, polyropylen, teflon, polyethyleneterephthalate, polyvinylidenchloride, PVC, polyimide, PVA, polycarbonate.

The term “magnetic bead” refers to a particle composed of a magnetic or ferromagnetic inner core and an outer solid matrix shell, which is attracted when a magnetic field is applied.

The preferred embodiment of the material according to the invention is selected from a group consisting of:

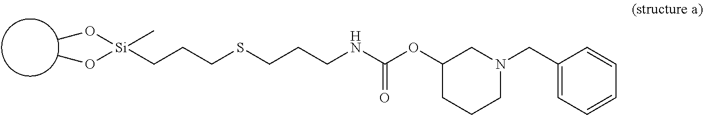

3-(allylcarbamoyloxy)-N-benzylpiperidine immobilized onto 3-mercaptopropylsilica matrix

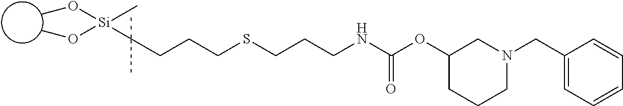

The substructure on the left hand side represents the supporting matrix and the substructure on the right hand side of the dotted line represents the ligand.

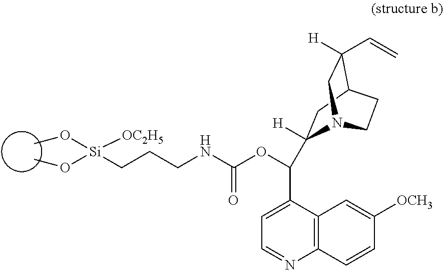

triethoxysilyl-activated propylcarbamoylquinine-ligand immobilized onto a bare silica matrix

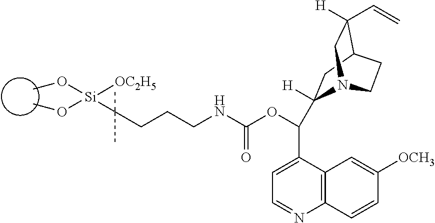

The substructure on the left hand side represents the supporting matrix and the substructure on the right hand side of the dotted line represents the ligand.

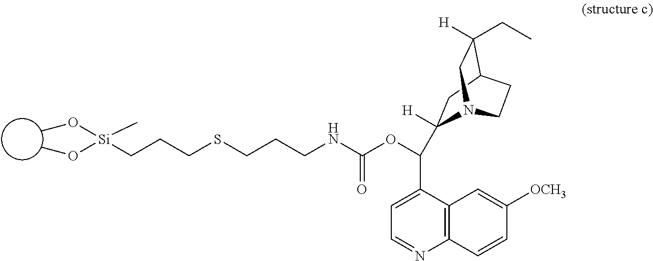

allylcarbamoyl-10,11-dihydroquinine ligand immobilized onto a 3-mercaptopropyl-modified silica matrix.

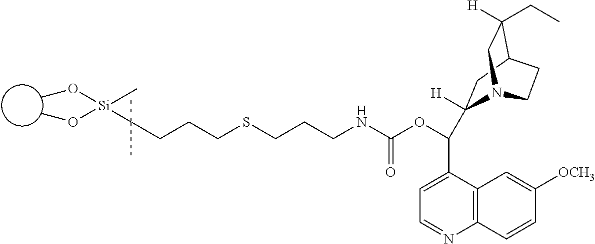

The substructure on the left hand side represents the supporting matrix and the substructure on the right hand side of the dotted line represents the ligand.

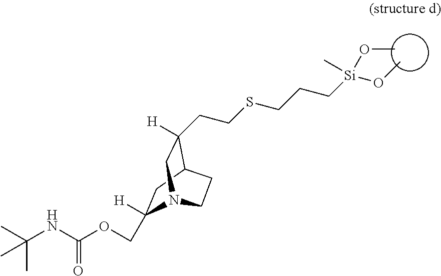

O-tert-butylcarbamoyl-quincorine immobilized onto 3-mercaptopropyl-modified silica matrix

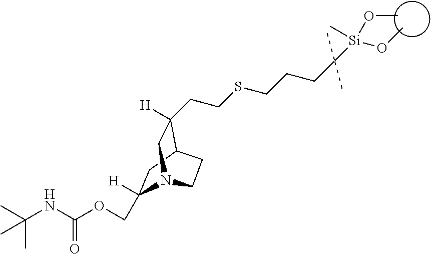

The substructure on the right hand side represents the supporting matrix and the substructure on the left hand side of the dotted line represents the ligand.

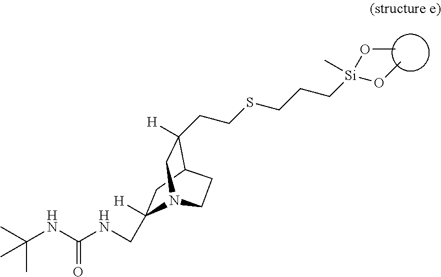

tert-butylquincorinyl-urea ligand immobilized onto a 3-mercaptopropyl-modified silica matrix

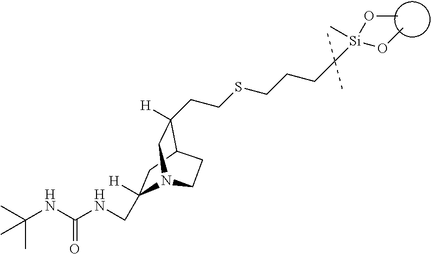

The substructure on the right hand side represents the supporting matrix and the substructure on the left hand side of the dotted line represents the ligand.

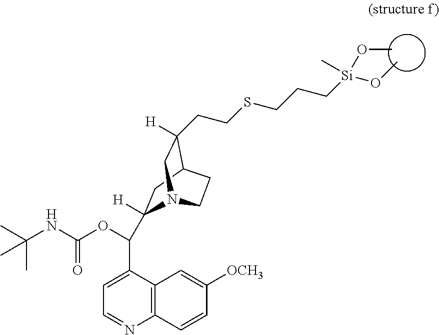

tert-butylcarbamoyl-quinine immobilized onto mercaptopropyl-modified silica matrix

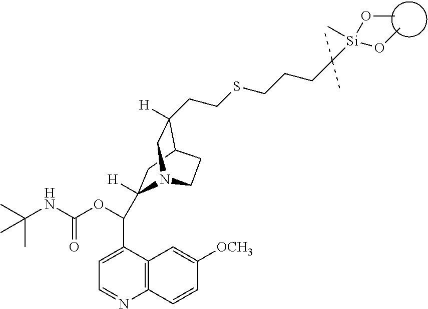

The substructure on the right hand side represents the supporting matrix and the substructure on the left hand side of the dotted line represents the ligand.

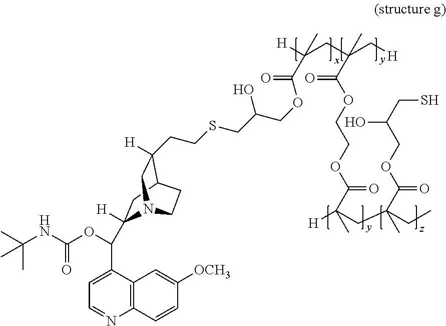

tert-butylcarbamoyl-quinine ligand immobilized onto thiol-modified polymethacrylate beads (Suprema-Gel 30u)

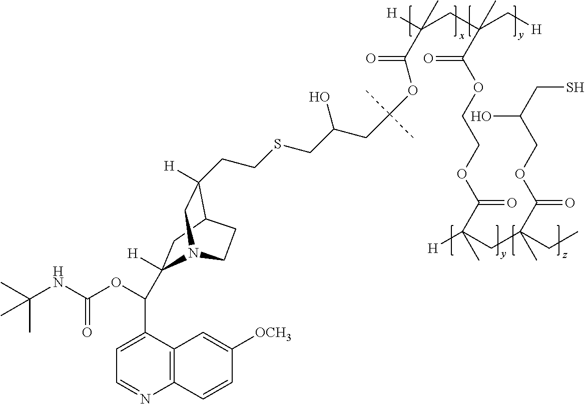

The substructure on the right hand side represents the supporting matrix and the substructure on the left hand side of the dotted line represents the ligand.

The separation of all three pDNA isoforms or topoisomers is governed by the material comprising carbamoyl-decorated anion-exchange ligands, which have not been employed so far for this type of liquid phase separation. These ligands according to the invention comprise a well defined arrangement of a NH-donor, H-acceptor, and the anion exchange site. Hence the plasmid isoforms/topoisomers interaction with the material comprising the ligand(s) is directed in a defined way, ensuring an invariant elution order. Thus, the present invention claims a new reliable and predictable concept for separation of very large biomolecules based on chemoaffinity principles. Furthermore, the ccc topoisomers have a potential to represent an additional quality control parameter in the biotechnological production including the final product characterization.

It will be apparent to those skilled in the art that various modifications can be made to the materials, methods and processes of this invention. Thus, it is intended that the present invention covers such modifications and variations, provided they come within the scope of the appended claims and their equivalents. All publications cited herein are incorporated in their entireties by reference.

Chromatographic ligands suitable for plasmid DNA separation are designed in order to make an easy attachment to the matrix possible (Example 1), wherein a ligand containing a terminal alkene group reacts with a thiol group in a radical addition reaction. Thus, in a first step a linker is attached to the matrix containing a terminal thiol group. In a second step, the matrix and the alkene-containing ligand are mixed in the presence of a radical initiator to give a stable thio-ether bond between the ligand and the linker bound to a matrix (see structure a). Using this process, stable chromatographic materials are easily obtained with a high ligand coverage on the matrix surface.

To practically assess the role of the materials according to the invention two of quinine-carbamate ligands, namely propylcarbamoylquinine and allylcarbamoyl-10,11-dihydroquinine, are employed for the chromatographic analytical separation of plasmid pMCP1 (Boehringer Ingelheim, size 4.9 kb). The first one is anchored via a short propyl-linker between the ligand and the silica matrix (4 bonds between the first silicon atom of the matrix and the NH hydrogen donor, see structure b), and the other one via a long linker (8 bonds between the first silicon atom of the matrix and the NH hydrogen donor, see structure c) to the thiopropyl-modified silica matrix. Comparison of the chromatographic properties of the long linker and the short linker disclose that the recovery of the oc form is significantly improved by use of the short-linked ligand (i.e. full recovery equal to 1 μg of the 4.9 kb plasmid is recorded during one chromatographic run as shown in Example 2). The main difference is the length of the linker (3 atoms versus 7 atoms), i.e. the distance between the ligand and the surface of the matrix is not identical. Using the material containing the short linker complete recoveries of all isoforms including the oc form are found due to proximity of negatively charged remaining silanols which have a repulsing effect on pDNA. Furthermore, these data show overall improvement (superiority) of the material properties in terms of resolution between oc and ccc form as well as in terms of oc recovery of the material when compared to the commercially available columns currently used for pDNA isoform analysis.

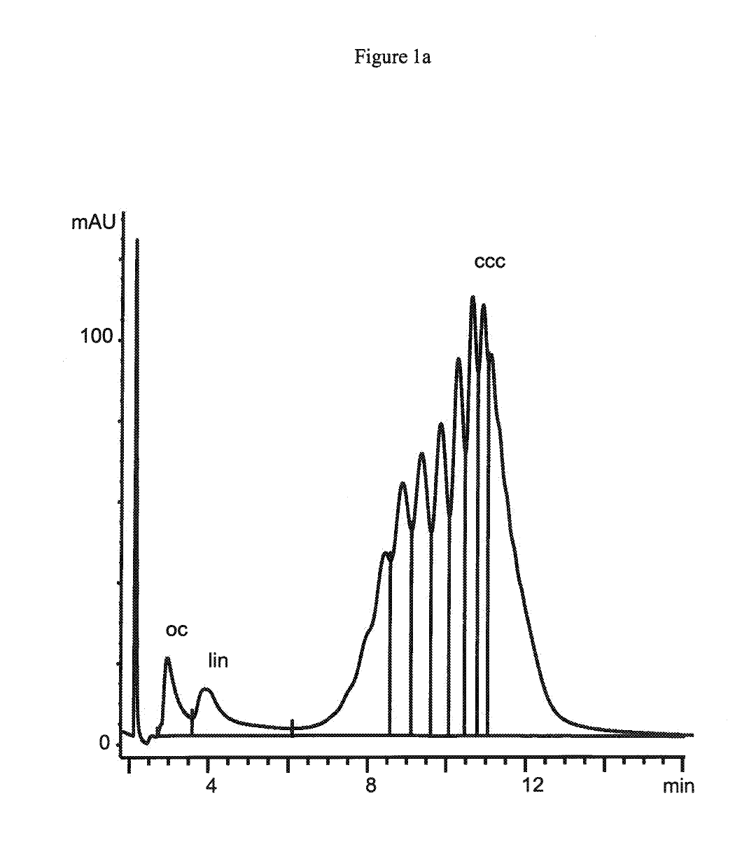

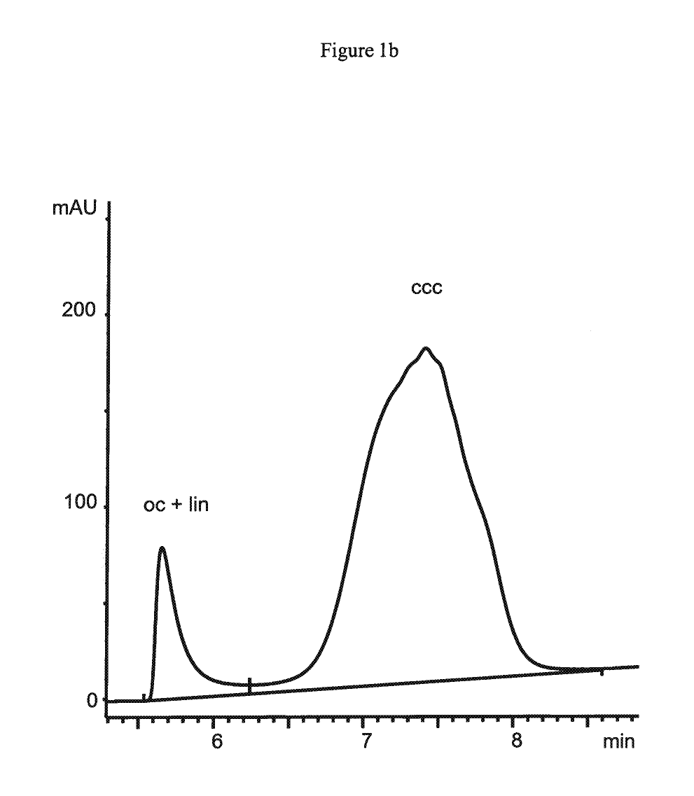

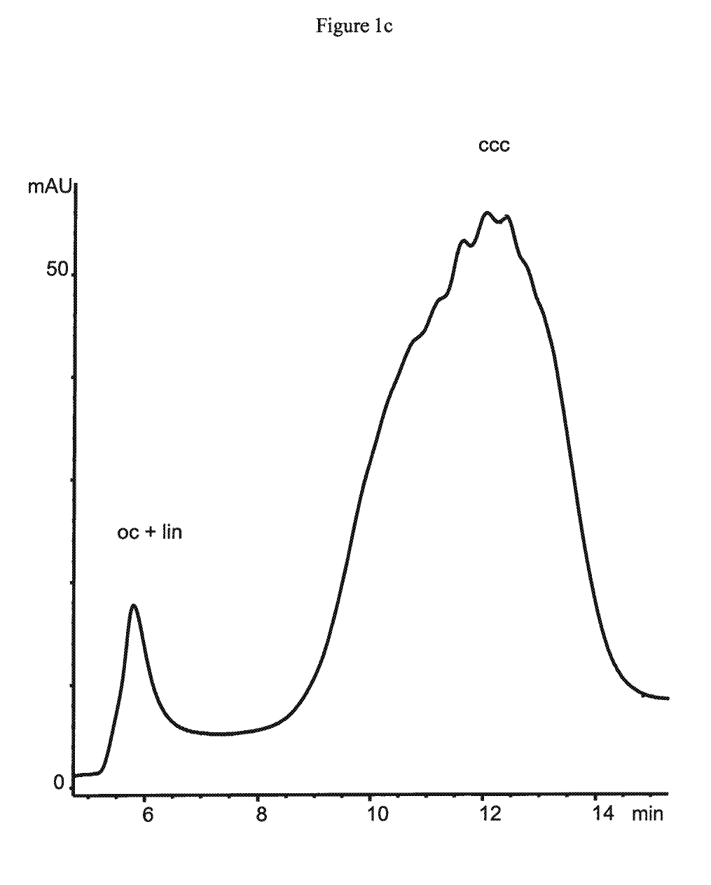

To test the impact of the morphology of the matrix, the long-linked ligand described in the previous paragraph is attached via the long linker to the following silica-based matrixes: 1.5 μm non-porous particles, 10 μm porous particles and a monolith (Example 3). Columns containing these materials are tested in the analogical experimental setup as disclosed in the previous paragraph. Here, the samples containing all three isoforms of pDNA (oc, ccc and lin isoforms of the pMCP1 plasmid) are loaded onto the columns and eluted from the column under identical conditions. Substantial differences are observed between these solid supports. All tested matrixes are suitable for separating the ccc isoform from the oc isoform and for separating the ccc isoform from the lin isoform. Additionally, when using the 1.5 μm matrix, an efficient separation between the oc and linear isoforms is achieved (see in FIG. 1a). Thus, for analytical purposes the 1.5 μm support is the preferred choice. However, the 10 μm porous matrix (see FIG. 1b) and the monolithic support (see FIG. 1c) both are suitable for preparative applications focusing merely on the separation of ccc form. In spite of the coelution of the oc and lin isoforms, still effective separation of the ccc form is achieved in this case.

Concerning the topoisomer separation, chromatographic selectivity calculated as retention time difference between the highest abundant topoisomers is found to be equal (0.12±0.02) on all tested supports. This shows the importance of the chemoaffinity separation principle resulting in high robustness of the separation. However, the 1.5 μm matrix provides the highest separation efficiency due to small peak width and is thus the preferred choice for analytical purposes.

In general, it was found that small non-porous spherical particles provide best separation efficiencies for pDNA isoforms and topoisomers and are thus most suitable for analytical scale separation. Large porous particles and monoliths still provide good separation of pDNA forms, especially when separating the ccc isoform from the oc and linear isoforms. Due to their low pressure drops and higher binding capacities, especially inherent to monoliths, they are more suitable for preparative applications.

Furthermore, Example 4 compares two different methods for pDNA separation, namely Method 1 by using of the increasing gradient of sodium chloride (NaCl) and Method 2 with the increasing pH gradient. In both cases a chromatographic column containing the material based on 1.5 μm non-porous particles (structure b), is applied for pDNA separation. Significant differences are found between these two analytical methods for pDNA characterization. During analysis using Method 1 with a combined gradient of NaCl and of 2-propanol for the elution of the bound pDNA, the mobile phase concentration of both, NaCl and 2-propanol increases simultaneously. In this case the oc, the linear and the individual ccc topoisomers are all separated during a single chromatographic run (see FIG. 1a).

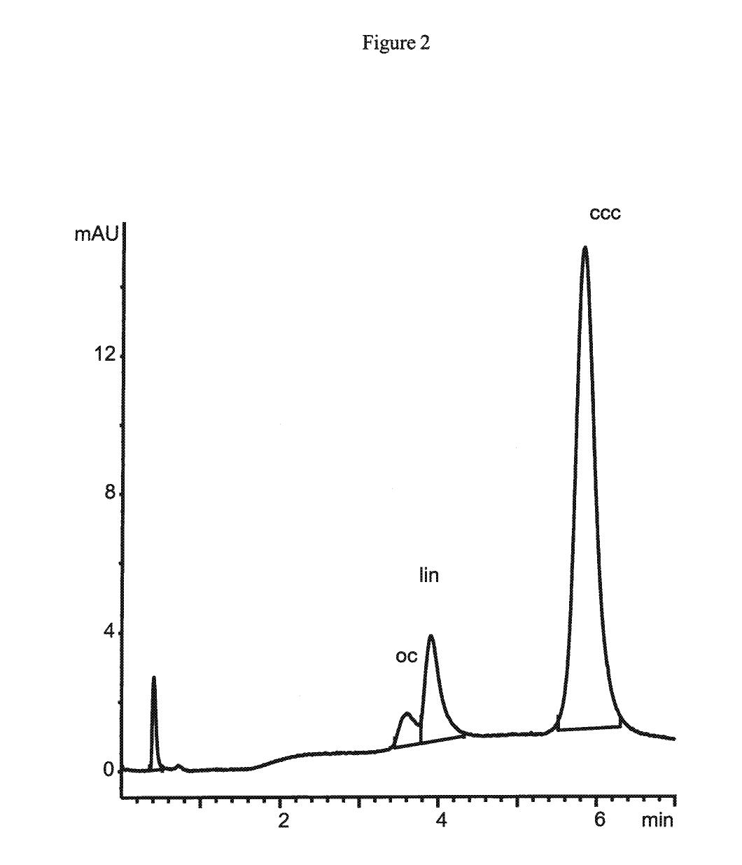

During analysis using Method 2 with a combined gradient of pH and of 2-propanol for the elution of the bound pDNA, the pH value of the mobile phase concentration and its 2-propanol contents increase simultaneously. In this case a separation of all three isoforms (oc, ccc and linear) is achieved (see FIG. 2a), however lacking the separation of the individual topoisomers. Since the ccc isoform is eluted as a single peak, the separation of the other two forms is further improved compared to NaCl-mediated elution. Additionally, when 1 μg of each isoform is loaded onto a 50×4.6 mm column, full recoveries of all isoforms (oc, lin and ccc) in the whole range of tested plasmid sizes, namely 4.9 kb and especially 9.9 kb and 14.5 kb) are found (i.e. the separation of large-sized pDNA separation is superior when compared to the known techniques such as ion exchange chromatography). Therefore, Method 2 is preferred for the analytical determination of the plasmid homogeneity, i.e. the content of the ccc form relatively to the other forms (oc and lin), independent on the size of the plasmid, as well as for preparative application for isolation of ccc pDNA (without topoisomer separation), due to its narrow peak form compared to NaCl-mediated elution and lower salt loads of the collected fractions.

To demonstrate the variability of ligands to be used for pDNA isoform and topoisomer separation, three different ligands are attached to 5 μm spherical silica particles, namely tert-butylcarbamoyl-quincorine (structure d) and tert-butylquincorinyl-urea (structure e), both attached via a long linker, and propylcarbamoylquinine attached directly (structure b) to the support (Example 5). The ccc pDNA sample containing about 5% of the oc isoform is loaded onto each of these columns and eluted under identical conditions employing a linear gradient of sodium chloride and 2-propanol. FIG. 3a shows a chromatographic separation of such pDNA sample employing a tert-butylquincorinyl-urea ligand and FIG. 3b shows such separation employing a tert-butylcarbamoyl-quincorine ligand, while in FIG. 4a (continuous line) such separation is shown employing a propylcarbamoylquinine ligand. On all these columns, a comparable elution pattern (equal elution order) is obtained demonstrating the robustness of the chemoaffinity principle. Firstly, the oc isoform elutes from the column, after which the so called Boltzmann-distributed pattern of ccc topoisomers is obtained (i.e. relative concentrations of each individual species according to their free energy; probability measure for the distribution of the states of a system according to the energy). Such a novel and robust separation, especially of plasmid topoisomers, represents a major invention for the use of plasmid DNA and depicts a significant advantage for both, analytical and preparative scale applications.

In order to demonstrate the loading capacity of the material, a total pDNA amount of 284 μg is loaded onto a 50×4.6 mm column containing 1.5 μm non-porous silica support with a propylcarbamoylquinine ligand. For this experiment the maximum injection volume of the HPLC system is employed. Upon elution with a combined salt and 2-propanol gradient, a chromatogram is obtained as shown in FIG. 3c. No overloading effects and no reduction of the chromatographic resolution between the individual topoisomer peaks are observed. Thus, by use of a single analytical column of such small dimensions, milligram quantities of an individual topoisomer can easily be isolated after approximately 30 to 50 runs. Furthermore, chromatography in general is characterized by the linear scalability, thus larger-scale preparative applications can be set up following general rules for chromatographic upscaling.

The elution pattern of topoisomers is assessed by analyzing the eluted fractions using a complementary method, capillary gel electrophoresis (Example 6). During the elution of individual topoisomers, it is found that those which are being less negatively supercoiled elute firstly. Accordingly, topoisomers with a higher negative supercoiling elute later, shown by separating the isolated chromatographic fractions “A” and “B from the chromatogram in FIG. 4a (dashed lines) by chloroquine-containing capillary gel electrophoresis (FIG. 4b). The less supercoiled topoisomer, in this example topoisomer “A”, has also lower writhe, thus the molecule is of bigger size than the more supercoiled topoisomer “B. Since the gel electrophoresis separation is size-dependent, the topoisomer “B migrates faster than the topoisomer “A”, and the relaxed oc isoform is the slowest migrating species in the sample. The topoisomer patterns for the pMCP1 plasmid (4.9 kbp) following the Boltzmann distribution, are also recorded employing the column containing a propylcarbamoylquinine ligand by analyzing commercially available plasmids such as pUC19 (2.7 kbp, Sigma Aldrich), pBR322 (4.4 kb, Sigma Aldrich), pET-40b(+) (6.2 kbp, Invitrogen), or pBACsurf-1 (9.5 kbp, Invitrogen).

In order to demonstrate the relevance of the topoisomer analysis, a plasmid topoisomer pattern is studied over the time course of the plasmid fermentation (Example 7). Cell mass samples containing bacterial cells are disrupted using NaOH/SDS. The suspension is centrifuged to obtain the cytoplasm content containing pDNA. Two-dimensional high-performance liquid chromatography (HPLC) approach is used, wherein in the first step a total pDNA is isolated from the mixture using size exclusion chromatography (SEC) and in the second dimension an allylcarbamoyl-10,11-dihydroquinine ligand-containing silica (shown in structure c) column is employed for ccc topoisomer separation. Fermentation samples, drawn in two-hour intervals, are analyzed by 2D HPLC. The results are shown as an overlay of chromatograms obtained after the second dimension (FIG. 5a). The chromatograms shown are recorded from samples taken after 11, 13, 15 and 17 hours from the beginning of the fermentation. The maximum of the topoisomer distribution, represented by the most populated ccc topoisomer (marked with an arrow in FIG. 5a) is shifting during the fermentation. In this case the overall supercoiling shifts towards lower negative supercoiling. The entire progression of the distribution maximum is shown in FIG. 5b. During the fermentation process the supercoiling of ccc pDNA changes significantly during the first hours of the fermentation but remains similar towards the end. These data support the importance of the topoisomer analysis which can be used as an indicator of the process stage and as a quality control parameter determined in a simple way by using liquid chromatography, and for the stability studies over the whole production process.

### EXAMPLES

### Example 1

### Scheme for Synthesis of a Ligand and Coupling to a Solid Matrix

Scheme 1: 980 mg (5 mmol) of (R)-(−)-1-Benzyl-3-hydroxypiperidine (Sigma Aldrich) are transferred into a three-necked round bottom flask and dissolved in 15 ml dichloromethane. The apparatus is flushed with nitrogen, and 502 μl (5.7 mmol, 1.15 eq.) allylisocyanate (Fluka) together with 3 μl (5 μmol) dibutyltindilaurate (Aldrich) as a catalyst are added to the mixture. The yellowish solution is refluxed 3 hours and the reaction progress is monitored by tlc (dichloromethane: methanol=20:1). After flash silica column chromatography using dichloromethane:methanol=25:1 as eluent, the product, 3-(allylcarbamoyloxy)-N-benzylpiperidine, is isolated in 58% yield. 3-mercaptopropyl-modified silica matrix (or support) is produced from bare 5 μm spherical silica (30 g, Daiso, Japan) and 3-mercaptopropyl-methyldimethoxysilane (8.6 ml) by refluxing in dry toluene in the presence of 4-dimethylaminopyridine (57 mg, Fluka) for 7 hours. Endcapping is performed by refluxing the generated 3-mercaptopropyl-modified silica with 1,1,1,3,3,3-hexamethyldisilazane (Fluka) in toluene for 3 hours to form trimethylsilyl (TMS)-endcapped silanols. 330 mg (1.2 mmol) 3-(allylcarbamoyloxy)-N-benzylpiperidine and 2.05 g endcapped mercaptopropyl-modified 5 μm silica are transferred into a three-necked round bottom flask equipped with a mechanical stirrer and suspended in 30 ml methanol. The apparatus is vigorously flushed while adding a solution of 5 mg azo-bis-(isobutyronitril) (AIBN, Merck) in 5 ml methanol. The mixture is refluxed for 20 hours under nitrogen atmosphere, and then it is filtrated through a sintered glass filter (porosity 3). After washing the silica material well with methanol, it is dried at 60° C. for 48 hours. The synthesized material represents 3-(allylcarbamoyloxy)-N-benzylpiperidine immobilized onto 3-mercaptopropylsilica matrix (structure a).

Scheme 2: Thiol-modified organic polymer matrix is obtained as follows: reaction buffer is prepared by dissolving 178 mg disodiumhydrogenphosphate (Merck) in a mixture of 20 ml distilled water and 5 ml 2-propanol (Roth). The pH is adjusted to 8.0 with diluted orthophosphoric acid. 132.6 mg Suprema-Gel 30u (Polymer Standards Service-USA, Inc.) are suspended in 1.8 ml reaction buffer. Then, 192 μl of a solution of NaSH hydrate (Sigma Aldrich) in reaction buffer (100 mg/ml) is added, and the mixture is stirred for 2 hours at 63° C. in a thermomixer (Eppendorf). By means of a small glass funnel (porosity 3), the modified material is washed twice with reaction buffer, twice with water, once with 0.1 mol/l HCl, again with water, and twice with methanol.

Reduction of disulfides to terminal thiols: TCEP buffer is prepared by dissolving 672 mg sodiumdihydrogenphosphate in a mixture of 11.8 ml water and 2.8 ml methanol (measured pH is 4.6). Then, 19 mg (75 μmol) tris(2-carboxyethyl)-phosphine hydrochloride (TCEP) (Fluke) are dissolved in 1 ml of TCEP buffer. 34.5 mg of thiol-modified Suprema Gel is added to the solution and the mixture is stirred overnight at room temperature. The suspension is filtered through a small funnel and the material is washed with TCEP buffer, methanol and finally with hexane. Elemental analysis (54.52% carbon, 7.67% hydrogen, 1.40% sulphur) revealed a sulphur concentration of 0.44 mmol/g while no nitrogen is present. The concentration of reactive thiols is 0.14 mmol/g, as determined spectrophotometrically (Nogueira et al., Analytica Chimica Acta, 2005, vol. 533 (2), pp. 179-183).

Ligand-modification: 14.4 mg of reduced thiol-modified Suprema-Gel 30u are transferred into a safe-lock plastic reaction tube (eppendorf). 130 μl of a 3.2 mg/ml solution of tert-butylcarbamoylquinine (produced in-house) in methanol, 10 μl of a 2 mg/ml solution of AIBN (Merck) in methanol and 0.36 ml methanol are added and the mixture is purged with nitrogen. The reaction tube is stirred at 65° C. for 18 h. The suspension is filtered through a small funnel and the material is washed 3× with methanol and finally with petroleumether. Elemental analysis (50.27% carbon, 7.70% hydrogen, 0.11% nitrogen, 0.97% sulphur) reveals a successful immobilization of the tert-butylcarbamoylquinine ligand onto the thiol-modified organic polymer matrix (structure g).

### Example 2

### Impact of the Linker's Length on the Recovery of Plasmid Isoforms

Two solid supports for chromatography are synthesized bearing quinine-carbamate ligands, onto one of which the ligand is anchored via a short linker to the matrix (triethoxysilyl-activated propylcarbamoylquinine on bare silica, structure b), and onto the other matrix via a long linker (9-allylcarbamoyl-10,11-dihydroquinine on 3-mercaptopropyl-modified silica, see structure c). The support with the long linker is synthesized according to Example 1, Scheme 1, starting from 10,11-dihydroquinine (Buchler, Germany) and allylisocyanate (Sigma Aldrich) in 95% yield and attached to endcapped 3-mercaptopropyl-modified silica. The ligand density according to the elemental analysis (14.01% C, 2.18% H, 1.38% N, and 1.90% S) is 318 μmol/g.

For the production of the matrix bearing the short linked ligand, 3-isocyanatopropyl-triethoxysilane (1 mol eq.) and quinine (1.05 mol eq.) are refluxed in methanol to yield the carbamate-containing product. Bare 5 μm spherical silica is added directly to the mixture (0.5 g of bare silica per mmol of quinine) while stirring mechanically and the suspension is refluxed further to obtain triethoxysilylpropylcarbamoylquinine-modified silica. After washing with methanol, the silanol groups are endcapped according to example 1, scheme 1. The ligand density according to the elemental analysis (7.90% C, 1.21% H, 0.911% N) is 217 μmol/g.

HPLC columns containing the produced supports are packed in-house with the modified 5 μm silica stationary phases, at a pressure of 600 bar. HPLC analyses are carried out on an Agilent 1200 SL system (Waldbronn, Germany) equipped with a binary pump, a thermostatted autosampler (cooled to 4° C.) and a DAD UV detector using a D₂ lamp as UV source. Sample components are eluted by using Method 1, i.e. an increasing NaCl gradient and an increasing 2-propanol gradient simultaneously (“mixed NaCl and 2-propanol gradient”).

Eluents for HPLC and chromatographic conditions: Firstly, a 0.5 mol/L stock solution from NaH₂PO₄ (Merck) is prepared. Eluent A consisted of 1:10 diluted stock solution (50 mmol/L NaH₂PO₄) titrated to 7.0 with 5M NaOH. Eluent B consisted of 1:10 diluted stock solution (50 mmol/L NaH₂PO₄) containing 0.6 mol/L NaCl (Fluka) and 10% (v/v) isopropanol (Roth) titrated to 7.0 with 5M NaOH. A gradient from 0 to 100% B in 15 minutes is run during analysis. The flow rate is set to 0.7 ml/min, detection wavelength to 258 nm (slit: 4 nm, reference 360 nm with 100 nm bandwidth) and the temperature to 50° C. (with preheating of the solvent in the 3 μl heat exchanger).

For recording chromatographic runs, 3 μl of a 2.84 mg/ml solution of a ccc pDNA sample containing 5% oc form (based on results of gel electrophoresis) is loaded onto the column and eluted with increasing amount of eluent B. After washing the column by injecting 4 times a 50 μl plug of 3 M NaCl while washing the column at 100% eluent B to assure the absence of a carry-over and reequilibrating the column at 0% B for 5 minutes, 20 μl of a 0.05 mg/ml solution of the oc isoform is loaded onto the column and eluted with increasing amount of eluent B. After further washing the column by injecting 4 times a 50 μl plug of 3 M NaCl and washing the column at 100% eluent B, 20 μl of a 0.05 mg/ml solution of the lin isoform is loaded onto the column and eluted with increasing amount of eluent B. When comparing the peak areas obtained for the oc and the linear isoform using the matrix with the long-linked ligand, the recovery (calculated as the ratio between the peak area per μg pDNA of the oc isoform and the peak are of the linear isoform per μg pDNA) is about 60%. However, when using the short-linked ligand the data are in conformity with each other within the analytical error (5%). When comparing the peak areas obtained for the ccc and the linear isoform samples, both supports tested show recoveries between 95 to 100%.

### Example 3

### Impact of the Matrix Morphology on the Separation of pDNA Isoforms and Topoisomers

A tert-butylcarbamoylquinine ligand synthesized from tert-butylisocyanate and quinine in accordance to the procedure as described in Example 2 is attached via 3-mercaptopropylsilane activated support to 1.5 μm non-porous silica particles (obtained from Micra Scientific Inc., USA), to 10 μm porous silica particles (obtained from Daiso Co, Ltd., Japan) and a silica monolith Chromolith™ (obtained from Merck, Germany) containing 2 μm macropores. These supports have specific surface areas of 3 m²/g for the 1.5 μm particles and 300 m²/g for the 10 μm particles and the monolith, respectively. 1.5 μm non-porous silica particles are packed into a 50×4.6 mm column by Bischoff Chromatography (Germany), while the 10 μm particles are packed in-house at a pressure of 600 bar into a 150×4.0 mm column. The chromatographic equipment as well as the employed chromatographic conditions are disclosed in Example 2.

Samples containing all three isoforms are prepared by mixing 60 μl aqueous solution with 10 mM ethylenediaminetetraacetic acid disodium salt (“EDTA-Na₂”, Fluka), 10 μl of a 2.84 μg/μl ccc pDNA sample containing about 10% oc isoform, with 50 μl 0.05 μg/μl linear isoform. Linear isoform is generated by digestion with EcoR V (Sigma Aldrich) according to the manufacturer's instructions and EDTA is added for inactivation of the endonuclease. In the chromatographic runs, 10 μl of these pDNA sample solutions containing all three isoforms of pDNA (oc, ccc and lin isoforms of the pMCP1 plasmid) are loaded onto the columns and eluted under identical conditions. For the analytical purposes the 1.5 μm support is the preferred choice. However, both the 10 μm porous support (see FIG. 1b) and the monolithic support (see FIG. 1c) are suitable for preparative applications focusing merely on the separation of ccc form.

### Example 4

### Chromatographic Method Using a Mixed pH and 2-Propanol Gradient Elution (Method 2) in Comparison to Mixed NaCl/2-Propanol Gradient Elution (Method 1)

100 μl of a pDNA sample, containing 0.15 μg oc isoform, 0.3 μg linear isoform and 14.2 μg ccc isoform, are loaded onto a material comprising of a triethoxysilylpropylcarbamoylquinine ligand (see structure b) attached to 1.5 μm non-porous silica. Sample components are eluted using Method 2, i.e. an increasing pH gradient and an increasing 2-propanol gradient simultaneously (“mixed pH and 2-propanol gradient”) on an Agilent 1200 SL system recording the UV absorption at 258 nm.

Eluents for HPLC and chromatographic conditions: First, a 0.5 mol/L stock solution from NaH₂PO₄ (Merck, Darmstadt, Germany) is prepared. Eluent A consisted of 1:10 diluted stock solution (50 mmol/L NaH₂PO₄) titrated to 7.2 with 5M NaOH. Eluent B consisted of 1:10 diluted stock solution (50 mmol/L NaH₂PO₄) and 20% (v/v) 2-propanol titrated to 7.9 with 5M NaOH. A linear gradient from 0 to 100% B (corresponding to a gradient from pH 7.2 to pH 7.9) in 15 minutes is run during analysis. Between the analytic runs, the column is washed by a plug of sodium chloride (injection of 50 μl 3M NaCl (aq.)) while the mobile phase composition is kept at 80% B for 1 minute, followed by reequilibration to 0% B for 5 minutes. The flow rate is set to 0.7 ml/min, detection wavelength to 258 nm (slit 4 nm, reference 360 nm with 100 nm bandwidth) and the temperature to 60° C. (with preheating of the solvent in the 3 μl heat exchanger). When comparing these chromatograms to those obtained in Method 1 described in Example 2 (see FIG. 1a), the latter one is preferred for separation of pDNA topisomers. Method 2 is preferred for the analytical determination of the plasmid homogeneity, i.e. the content of the ccc form relatively to the other forms (oc and linear), as well as for preparative application for isolation of ccc pDNA (without topoisomer separation).

### Example 5

### Demonstration of Ligand Variation and High Loadability

Three solid supports for chromatography are synthesized. The tert-butylcarbamoyl-quincorine ligand (structure d) is synthesized according to Example 1, Scheme 1, starting from quincorine (Buchler, Germany) and tert-butylisocyanate and performing flash silica column chromatography in ethyl acetate (redistilled in-house)/triethylamine (Fluka) (10:1) in 96% yield and attached to endcapped mercaptopropyl-modified 5 μm silica particles. The ligand density according to the elemental analysis (10.51% C, 1.98% H, 0.979% N, 1.85% S) is 307 μmol/g.

The tert-butylquincorinylurea ligand (structure e) is synthesized according to Example 1, Scheme 1, starting from quincorine-amine (Buchler, Germany) and tert-butylisocyanate and performing flash silica column chromatography in ethyl acetate (redistilled in-house)/methanol/triethylamine (Fluka) (10:1:1) in 96% yield and attached to endcapped 3-mercaptopropyl-modified 5 μm silica particles. The ligand density according to the elemental analysis (8.965% C, 1.87% H, 0.979% N, 1.94% S) is 210 μmol/g.

The triethoxysilylpropylcarbamoylquinine-modified silica matrix is produced according to Example 2, using 1.5 μm non-porous silica particles (Micra Scientific Inc., USA). The ligand density according to the elemental analysis is 9 μmol/g due to 100× smaller specific surface area. The triethoxysilylpropylcarbamoylquinine-modified silica material is packed into 4.6×50 mm columns, while the other two materials are packed into 150×4.0 mm columns.

Eluents for HPLC and chromatographic conditions: Firstly, a 0.5 mol/L stock solution from NaH₂PO₄ (Merck) is prepared. Eluent A consisted of 1:10 diluted stock solution (50 mmol/L NaH₂PO₄) titrated to 7.0 with 5M NaOH. Eluent B consisted of 1:10 diluted stock solution (50 mmol/L NaH₂PO₄) containing 0.6 mol/L NaCl (Fluka) and 30% (v/v) isopropanol (Roth) titrated to 7.0 with 5M NaOH.

A gradient from 60 to 100% B in 24 minutes (tert-butylcarbamoyl-quincorine and tert-butylquincorinylurea ligand) or 0 to 25% B in 15 minutes (triethoxysilylpropylcarbamoylquinine ligand) is run during analysis. The flow rate is set to 0.7 ml/min, detection wavelength to 258 nm (slit: 4 nm, reference 360 nm with 100 nm bandwidth) and the temperature to 60° C. (with preheating of the solvent in the 3 μl heat exchanger).

For recording chromatographic runs (FIGS. 5c, 5d and 6a), 3 μl of a 2.84 mg/ml solution of a ccc pDNA sample solution containing 5% oc form is loaded onto the column and eluted with increasing amount of eluent B. For demonstrating high loadability, 100 μl of the sample solution containing 284 μg total pDNA are loaded onto the 4.6×50 mm column and eluted with increasing amount of eluent B. Roughly estimated, the column contains about 0.5 g of the chromatographic material which is found to be sufficient for separating 284 μg total pDNA. All ligands according to the invention are able to separate pDNA isoforms and topoisomers confirmed by the same elution pattern, thus demonstrating the robustness of the chemoaffinity principle.

### Example 6

### Demonstration of Topoisomer Identity and its Elution Order

A matrix containing an allylcarbamoyl-10,11-dihydroquinine ligand (see FIG. 2b) is synthesized according to example 1, scheme 1 and packed in-house into 150×4.0 mm columns. The chromatographic system and chromatographic conditions are as described in example 5, except for the gradient which was run from 10 to 60% B in 30 min.

For recording chromatographic runs, 100 μl of a 2.84 mg/ml solution of a ccc pDNA sample containing about 4% oc form is loaded onto the column and eluted with increasing amount of eluent B. During the run, two fractions are collected after the UV detector each containing a single topoisomer. The fractions are dialyzed against purified water using a VSWP cellulose ester membrane (25 mm diameter, 25 nm pores) supplied by Millipore (MA, USA). The discs are put on the water surface with the shiny side up and a volume of 300 μl is loaded onto the disc surface carefully. After floating for 15 to 30 minutes at 4° C., the samples are recovered with a pipette and 20 μl of this solution from each fraction is re-injected into the chromatographic system.

Capillary gel electrophoresis is performed in a ^{3D}CE instrument from Agilent Technologies. A DB-17 coated capillary with 100 μm i.d. is purchased from J&W Scientific (Folsom, Calif.), cut to a length of 32 cm and a detection window is made by removing the polyimide coating with a razor blade (effective length 24.5 cm). For topoisomer analysis, the capillary is filled with Tris-borate-EDTA (TBE, 89 mM boric acid, 89 mM Tris, 2 mM EDTA, titrated to pH 9.0 with NaOH) buffer containing 0.1% hydroxypropylmethylcellulose (HPMC, 86 kDa) purchased from Acros organics (Geel, Belgium). For complete homogenization of the buffer, the solution is stirred for 24 h after addition of the biopolymer and afterwards let to stand for another 24 h without disturbing at room temperature. For gaining higher resolution of highly supercoiled ccc plasmids, an intercalator is added to the electrophoresis buffer [de Carmejane et al., Proceedings of SPIE-The International Society for Optical Engineering, 1999, vol. 3602, p. 346-354], in this case 12 μl of a 5 mg/ml aqueous chloroquine diphosphate (Fluka) solution to 4 ml of electrophoresis buffer to give a final concentration of 15 μg/ml chloroquine, respectively. Samples are introduced by electrokinetic injection at −5 kV for 4 seconds. Electrophoresis is then performed in negative mode at 3.3 kV for 25 minutes at 25° C., with UV detection at 258 nm. Before injection, the capillary is flushed with water for 3 minutes and then preconditioned with running buffer for 5 minutes. Overall, during the elution of individual topoisomers, it is found that those which are being less negatively supercoiled elute firstly.

### Example 7

### In-Process Control of Topoisomer Distribution During Biotechnological Production of Plasmid DNA

During a batch fermentation of a pDNA encoded as AS24 (4.4 kb, Boehringer Ingelheim) in E. coli, samples are drawn in 2 hour intervals and frozen immediately. Before analysis, the samples are thawed and the pDNA is isolated and concentrated using a MiniPrep Kit (Qiagen) according to the manufacturer's instructions. The crude plasmid samples are directly injected into a two-dimensional HPLC system and analyzed.

Chromatographic conditions: 2D HPLC system (biocompatible Ultimate 3000 by Dionex), containing two additional 10-1 column switching valves

1st dimension: SRT SEC 1000 (size exclusion column) by Sepax Technologies Inc. (USA), Eluent: 0.1 mol/l Tris (Fluke) with 0.1 mol/l NaCl and 1 mmol/l EDTA, pH 7.5 (isocratic elution), flow rate: 2.0 ml/min

2nd dimension: 100×4.0 mm column with 5 μm silica containing ligand as disclosed in FIG. 2b, Eluent A: 50 mM phosphate buffer pH 7.2, Eluent B: Eluent A with 0.6 mol/l NaCl and 10% (v/v) isopropanol pH 7.2, column temperature 60° C., flow rate 1.0 ml/min, det. UV 258 nm; linear gradient 0-100% B in 15 min, inj. vol. 20 μl. During the fermentation process the supercoiling of ccc pDNA changes significantly, which is reliable determined by using the method of separation of pDNA topoisomers according to the invention.

### Example 8

### Cinchorine-Type Ligand on CIM Monolith

A CIM® Epoxy Disk Monolithic Column (BIA Separations) is rinsed with a freshly prepared 2 M solution of sodium hydrogen sulfide (Sigma-Aldrich) in a mixture of methanol and 0.1 M aqueous sodium dihydrogenphosphate (Merck), (20:80, v/v) (pH 8.15) in flow through-mode with an HPLC pump for 2 h. Afterwards, the column is washed with methanol/water (20:80, v/v) and then with methanol.

A 0.25 M solution of tert-butylcarbamoylquincorine (structure d) is prepared by dissolving it in methanol, and α,α′-azoisobutyronitrile (AIBN) (6 mg/ml, 0.037 M) is added as radical initiator. The mixture is sonicated for 5 min, filtered through a Nylon membrane, and purged with nitrogen for 10 min. Then, this tert-butylcarbamoylquincorine-solution is pumped into the thiol-functionalised CIM disc column. The column is sealed at both ends and transferred to a water bath, where the radical addition of the chromatographic ligand occurred at 60° C. After 24 hours the column is removed from the water bath, rinsed with methanol and then equilibrated with mobile phase using an HPLC pump. The separation of the plasmid isoforms is carried out using elution Method 1 (NaCl-gradient) with organic matrix, particularly suitable for preparative purposes.

### Example 9

### Modification of Epoxy-Group Containing Non-Porous Organic Polymer Particles

Epoxy-modified nonporous polymethacrylate beads (epoxy-NPR, 2.5 μm from Tosoh) are suspended in methanol in a three-necked round bottom flask. A 2 M solution of sodium hydrogen sulfide in a mixture of methanol and 0.1 M aqueous sodium dihydrogenphosphate, (20:80, v/v) (pH 8.15) is added (10-fold molar excess related to epoxy groups) and stirred under nitrogen flow for 2 h. Afterwards, the particles are filtered and washed with methanol/water (20:80, v/v) and then with methanol. A 0.25 M solution of tert-butylcarbamoylquincorine (structure d) is prepared by dissolving it in methanol, and α,α′-azoisobutyronitrile (AIBN) (6 mg/ml, 0.037 M) is added as radical initiator. The mixture is sonicated for 5 min, filtered through a Nylon membrane, and purged with nitrogen for 10 min. Then, this tert-butylcarbamoylquincorine-solution is added to a suspension of the above synthesized thiol-modified NPR particles in the three-necked round bottom flask. The radical addition reaction for bonding of the selector to the thiol-functionalised beads is carried out by stirring at 60° C. for 24 hours under a stream of nitrogen. Then, the particles are filtered and washed several times with methanol.

The particles are slurry packed into stainless steel column of the dimension 33×4.6 mm ID. The testing of the column for the separation of the plasmid isoforms is carried out using elution Method 1 (simultaneous NaCl- and 2-propanol gradients) with organic matrix, particularly suitable for analytical purposes.

1. A material for separation of plasmid DNA isoforms or topoisomers comprising a ligand according to formula 1 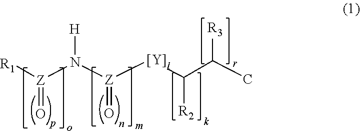
wherein the ligand contains a cationic group (C) and a hydrogen donor group (N—H) connected by a spacer with a length between 3 to 5 atoms,
m, o and r are independently from one another either 0 or 1, and
k, l, n and p are independently from one another either 0, 1 or 2, and
Y is any moiety selected from the group —CH₂—, —O—, —NH— or —S—,
Z is any moiety selected from the group —C—, —S—, or —P—, and
R₁, R₂ and R₃ are anyone of the substituents from the group consisting of hydrogen, C₁₋₁₈ branched or unbranched alkyl, C₂₋₁₈ branched or unbranched alkenyl, C₂₋₁₈ branched or unbranched alkynyl, C₃₋₁₁-carbocycle, C₃₋₈-heterocycle, C₅₋₁₈-aryl and C₅₋₁₃-heteroaryl,
wherein R₁, R₂ and R₃ optionally comprise independently from one another one or more moieties selected from the group —S—, —O—, —NH—, and
each of R₁, R₂, R₃ can be optionally and independently substituted with one or more substituents selected from the group consisting of hydrogen, C₁₋₆ branched or unbranched alkyl, C₂₋₆ branched or unbranched alkenyl, C₂₋₆ branched or unbranched alkynyl, C₃₋₈-carbocycle, C₃₋₈-heterocycle and C₅₋₁₀-aryl, C₅₋₁₀-heteroaryl, and
whereby the cationic group C is a C₃₋₁₁ heterocycle comprising at least one nitrogen atom and optionally another heteroatom selected from the group consisting of sulphur, oxygen, nitrogen and phosphorus, or
C₅₋₁₈ heteroaryl comprising at least one nitrogen atom and optionally another heteroatom selected from the group consisting of sulphur, oxygen, nitrogen and phosphorus, or
branched or unbranched C₁₋₁₀ alkyl, branched or unbranched C₂₋₁₃ alkenyl, branched or unbranched C₂₋₁₀ alkynyl comprising at least one nitrogen atom and optionally another heteroatom selected from the group consisting of sulphur, oxygen, nitrogen and phosphorus,
wherein C and R₃ or C and R₂ are optionally linked to each other by forming a ring, and
wherein the ligand is optionally immobilized onto or embedded into a matrix via R₁ or R₃ group.

2. The material according to claim 1 wherein the spacer length is equal to 4 atoms.

3. The material according to claim 1 wherein R₁ is anyone of the substituents selected from the group consisting of hydrogen, C₁₋₈ branched or unbranched alkyl, C₂₋₈ branched or unbranched alkenyl and C₂₋₈ branched or unbranched alkynyl, optionally comprising one or more sulphur atoms.

4. The material according to claim 1 wherein R₁ is anyone of the substituents selected from the group consisting of C₃₋₈-carbocycle, C₅₋₈-heterocycle, C₅₋₁₀-aryl and C₅₋₉-heteroaryl.

5. The material according to one of claim 1 wherein R₃ is anyone of the substituents selected from the group consisting of hydrogen, C₁₋₈ branched or unbranched alkyl, C₂₋₈ branched or unbranched alkenyl and C₂₋₈ branched or unbranched alkynyl, optionally comprising one or more sulphur atoms.

6. The material according to claim 1 wherein R₃ is a C₅₋₁₀ aryl or C₅₋₁₀ aryl heteroaryl.

7. The material according to claim 1 wherein the hydrogen donor group (N—H) is located adjacent to at least one hydrogen acceptor selected from a group consisting of carbonyl (C═O), sulfonyl (—SO₂—) and phosphonyl group (—P(═O)—).

8. The material according to claim 7 wherein the hydrogen acceptor is carbonyl group (C═O).

9. The material according to claim 1 wherein o=0 and m=1.

10. The material according to claim 1 wherein o=1 and m=0.

11. The material according to claim 1 wherein o=0 and m=1, and wherein k=1 and l=1, Z is —C— and n=1.

12. The material according to claim 1 wherein o=1 and m=0, and wherein k=1 and l=1, Z is —C— and p=1.

13. The material according to claim 1 wherein the matrix is solid or liquid, and selected from organic or inorganic polymers, wherein the inorganic polymer is silica based or zirconium oxide, and wherein the organic polymer is polymethacrylate or agarose based.

14. The material according to claim 13 wherein the matrix is a particle, a monolith resin, a membrane, or a magnetic bead.

15. The material according to claim 1 wherein the plasmid DNA isoforms are covalently closed circular (ccc), open circular (oc) or linear (lin) forms.

16. The material according to claim 15 wherein the plasmid DNA isoforms comprise mixtures of monomers, dimers, trimers, tetramers, oligomers, or multimers,
wherein the oligomeric and multimeric plasmid DNA is in form of catenane or concatemer.

17. The material according to claim 1 wherein the material is selected from a group consisting of:

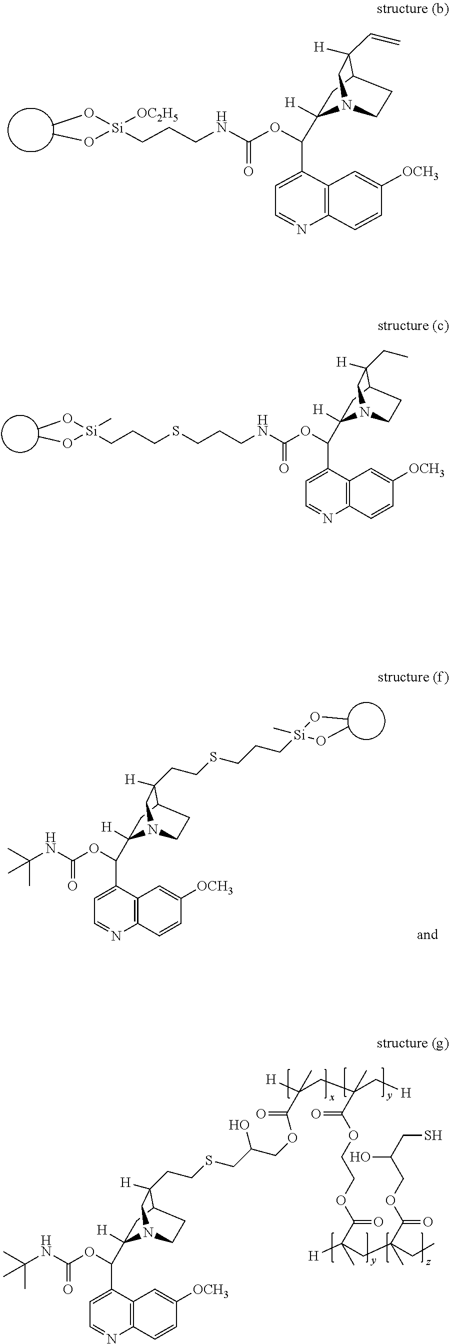

18. A material selected from a group consisting of:

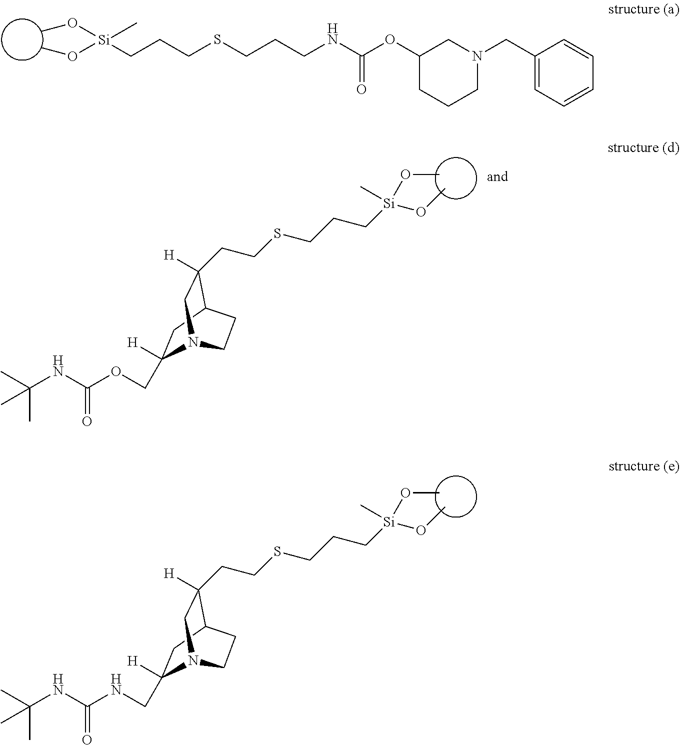

19. The material according to claim 18 for the separation of plasmid DNA isoforms or topoisomers.

20. A method for chromatographic separation of plasmid DNA isoforms or topoisomers comprising using the material according to claim 1.

21. A method for chromatographic separation of plasmid DNA isoforms or topoisomers comprising steps of:
a. dilution/dissolution of a sample containing pDNA isoforms or topoisomers;
b. loading of the sample onto the material of claim 1;
c. washing of the bound sample;
d. elution of the sample from material; and
e. detecting and/or collecting eluted fractions.
